(19) United States
(12) Patent Application Publication
(10) Pub. No.: US 2024/0261461 A1
(43) Pub. Date: Aug. 8, 2024

(54) SYSTEM AND METHODS FOR USING TISSUE-ADHESIVE POROUS HEMOSTATIC PRODUCTS WITH MINIMALLY INVASIVE SURGICAL TECHNIQUES

(71) Applicants: Ethicon, Inc., Somerville, NJ (US); Cilag GmbH International, Zug (CH)

(72) Inventors: Stuart HEAD, Zug (CH); Johannes Caspar Mathias Elizabeth BENDER, Nijmegen (NL); Edwin Alexander ROOZEN, Zug (CH); Erez ILAN, Zug (CH); Rosa Pilar FÉLIX LANAO, Zug (CH); Abraham KEEREWEER, Zug (CH); Gerard LLANOS, Somerville, NJ (US); Joost OPSTEEN, Zug (CH)

(21) Appl. No.: 18/431,414

(22) Filed: Feb. 2, 2024

Related U.S. Application Data
(60) Provisional application No. 63/483,044, filed on Feb. 3, 2023.

(51) Int. Cl. A61L 17/08

(52) U.S. Cl. CPC A61L 17/08; A61L 2400/04

(57) ABSTRACT

Methods and systems for using a biocompatible and flexible hemostatic sheet comprising a fibrous carrier structure, and reactive electrophilic groups capable of reacting with amine groups in tissue and blood, the hemostatic sheet delivered through a trocar for restoring hemostasis to a tissue at a bleeding site of an organ during minimally invasive treatment of hemorrhage.

### CROSS-REFERENCE TO RELATED APPLICATIONS

This application claims priority to U.S. Provisional Application Ser. No. 63/483,044, filed Feb. 3, 2023, the disclosure of which is hereby incorporated herein by reference in its entirety for all purposes.

### FIELD

This disclosure relates to devices and methods of applying medical products comprising biocompatible, covalently cross-linked polymers for reduction at bleeding sites.

### BACKGROUND

Hemostatic techniques are essential during surgery or other invasive procedures to provide hemostasis quickly and efficiently. Failure to achieve hemostasis can prolong surgery, impair wound healing, increase infection, and result in unanticipated exposure to blood products if the patient needs a transfusion. Both the short-term and long-term complications resulting from poor hemostasis, including excessive blood loss and allogenic blood transfusions during or after surgery, are associated with increased morbidity and mortality. Although hemostasis can be achieved with conventional techniques such as manual pressure, electrocautery, and ligature, these techniques may be ineffective in controlling bleeding from complex injuries and in less accessible areas.

If a residual bleeding persists despite application of conventional methods for hemorrhage control, adjunct hemostats can be applied in a variety of forms, including gels, patches, foams, and powders; however, these gels, patches, foams and powders can be difficult to deliver through a trocar and position at the site of bleeding during robotic surgical techniques.

In order for a topical hemostat to be effective in a minimally invasive situation, it must meet the following requirements: (1) be able to be introduced through a trocar, (2) be able to be successfully navigated towards a bleeding site, (3) be able to be successfully applied to a bleeding site to achieve hemostasis, and (4) should be able to handle up to at least moderate bleeding severity to prevent the need for conversion from minimally invasive to open surgery. Currently approved hemostatic agents used for open surgeries lack these abilities because they are either too stiff and are damaged during introduction, are not user-friendly and difficult to navigate to and position on the bleeding site, and/or are not effective enough on moderate bleedings and not suitable for severe bleeding.

In view of these clear performance disadvantages, there is a need for flexible, hemostats that can suitably be used to minimize hemorrhage during minimally invasive surgical procedures. The solution of this disclosure resolves these and other issues of the art.

### SUMMARY

The subject of this disclosure is the use of a biocompatible and flexible hemostatic sheet for restoring hemostasis to a tissue at a bleeding site of an organ during minimally invasive treatment of hemorrhage.

An example method of treating hemorrhage in a subject during a minimally invasive procedure can include delivering, through a trocar, a hemostatic patch near or about a bleeding site of an organ of a subject, positioning the hemostatic patch in contact with a tissue at the bleeding site, and restoring hemostasis to the tissue within three minutes or less. The hemostatic patch can include a fibrous carrier structure, and reactive electrophilic groups capable of reacting with amine groups in tissue and blood.

The present disclosure includes a method for treating hemorrhage in a subject during a minimally invasive procedure. The method can include delivering, through a trocar, a hemostatic patch near or about a bleeding site of an organ of the subject, positioning the hemostatic patch in contact with a tissue at the bleeding site of the organ, applying pressure to the hemostatic patch for approximately 30 seconds, and restoring hemostasis of the organ within approximately three minutes or less. The hemostatic patch can include a nucleophilic polymer carrying reactive nucleophilic groups, and an electrophilic polymer carrying at least three reactive electrophilic groups capable of reacting with the nucleophilic polymer and amine groups in tissue and blood.

The present disclosure includes a method for treating hemorrhage in a subject during a minimally invasive procedure. The method can include delivering, through a trocar, a first hemostatic patch near or about a bleeding site of an organ of a respective subject in a first plurality of subjects, positioning the first hemostatic patch in contact with a tissue at the bleeding site, applying pressure to the first hemostatic patch for approximately 30 seconds, and reducing time to hemostatic control of active bleeding from the bleeding site of the organ by delivering the first hemostatic patch to the first plurality of subjects compared to a second plurality of subjects treated by delivering a second hemostatic patch. The first hemostatic patch can include a nucleophilic polymer carrying reactive nucleophilic groups, and an electrophilic polymer carrying at least three reactive electrophilic groups capable of reacting with the nucleophilic polymer and amine groups in tissue and blood.

The present disclosure includes a device for treating hemorrhage in a subject during a minimally invasive procedure. The device can be a biocompatible, flexible hemostatic patch.

The hemostatic device can include a nucleophilic polymer carrying reactive nucleophilic groups, and an electrophilic polymer carrying at least three reactive electrophilic groups capable of reacting with the nucleophilic polymer and amine groups in tissue and blood. The hemostatic device can be capable of being delivered through a trocar to an organ of a subject and restoring hemostasis to the organ within approximately three minutes or less by positioning the hemostatic device in contact with tissue at a bleeding site of the organ.

The present disclosure includes a biocompatible, flexible, hemostatic device for treating hemorrhage in a subject during a minimally invasive procedure. The hemostatic device can include a water-resistant cohesive fibrous carrier structure. The fibrous carrier structure can include a three-dimensional interconnected interstitial space comprising a plurality of reactive polymer particles comprising an electrophilic polymer, and fibers comprising a nucleophilic polymer carrying reactive nucleophilic groups. The hemostatic device can be capable of being delivered through a trocar to an organ of a subject and restoring hemostasis to the organ within approximately three minutes or less by positioning the hemostatic device near or about a bleeding site of the organ.

To the accomplishment of the foregoing and related ends, certain illustrative aspects are described herein in connection with the following description and the appended drawings.

These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 shows a patient undergoing a laparoscopic procedure with an example hemostatic device delivered through a trocar and positioned at a bleeding site, in accordance with an embodiment of the present disclosure.

FIG. 2A shows a perspective view of an example hemostatic patch, in accordance with an embodiment of the present disclosure.

FIGS. 2B and 2C are structures of example reactive particles embedded in hemostatic patch, in accordance with an embodiment of the present disclosure.

FIG. 3 shows a summary of procedural times of an example of a study used for this disclosure of hemostatic patch versus suturing.

FIG. 4 shows a chart of surface bleeding severity scale (SBSS) scores verbal and visual descriptors and corresponding expected interventions.

FIG. 5 shows a summary of results from a superficial lesion of an example of a study used for this disclosure comparing hemostatic patch versus Comparative Device 1.

FIG. 6 shows a summary of results from a partial liver resection of an example of a study used for this disclosure comparing hemostatic patch versus Comparative Device 1.

FIG. 7 shows a summary of results from a deep metastasectomy of an example of a study used for this disclosure comparing hemostatic patch versus Comparative Device 1.

FIG. 8 is a table summarizing initial hemostasis between hemostatic patch and Comparative Device 2 and number of applications of each device, in accordance with an embodiment of the present disclosure.

FIG. 9 is a flowchart of an example method for treating hemorrhage in a subject during a minimally invasive procedure by delivering hemostatic patch, in accordance with an embodiment of the present disclosure.

FIG. 10 is a flowchart of an example method for treating hemorrhage in a subject during a minimally invasive procedure by delivering hemostatic patch, in accordance with an embodiment of the present disclosure.

FIG. 11 is a flowchart of an example method for treating hemorrhage in a subject during a minimally invasive procedure by delivering hemostatic patch, in accordance with an embodiment of the present disclosure.

FIG. 12 is a flowchart of an example method for treating hemorrhage in a subject during a minimally invasive procedure by delivering hemostatic patch, in accordance with an embodiment of the present disclosure.

FIG. 13 is a flowchart of an example method for treating hemorrhage in a subject during a minimally invasive procedure by delivering hemostatic patch, in accordance with an embodiment of the present disclosure.

FIG. 14 is a flowchart of an example method for treating hemorrhage in a subject during a minimally invasive procedure by delivering hemostatic patch, in accordance with an embodiment of the present disclosure.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

As used herein, the terms “about” or “approximately” for any numerical values or ranges indicate a suitable tolerance. More specifically, “about” or “approximately” can refer to the range of values ±20% of the recited value, e.g. “about 90%” can refer to the range of values from 71% to 99%.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, organ of a “subject” or “patient” may be an organ of a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

As discussed herein, “operator” may include a doctor, surgeon, or any other individual or delivery instrumentation associated with delivery of a clot retrieval device to the vasculature of a subject.

As discussed herein, “hemorrhage” can be understood as a release of blood from a broken blood vessel either inside or outside the body. The terms, “hemorrhage”, “bleeding site”, “rupture”, “blood flow”, “bleeding”, and/or the like, can be and are often used interchangeably throughout this disclosure.

The term “hemostatic patch” as used herein, unless indicated otherwise, refers to a sheet having the ability to stop bleeding from damaged tissue. The hemostatic patch of the present invention may achieve hemostasis by turning blood into a gel and/or by forming a seal that closes off the wound site.

The term “tissue-adhesive” as used herein refers to the ability of the hemostatic patch to cling to tissue due to the formation of covalent bonds between the sheet and the tissue. Formation of these covalent bonds typically requires the presence of water.

The term “water-resistant” or “insoluble” as used herein in relation to the fibrous carrier structure means that this structure is not water soluble and does not disintegrate in water to form a colloidal dispersion, at neutral pH conditions (pH 7) and a temperature of 37° C. In some examples, the fibrous carrier structure can absorb a lot of water such that it approaches or exceeds about 35 times its own weight.

The term “interstitial space” as used herein refers to the void (“empty”) space within the fibrous carrier structure. The interstitial space within the fibrous carrier structure allows the introduction of reactive polymer particles into the structure. Also, blood and other bodily fluids can enter the interstitial space, allowing the water-soluble electrophilic polymer within the reactive polymer particles to dissolve.

The concentration of reactive polymer particles having a diameter in the range of 0.5-100 μm is expressed in % by weight of the fibrous carrier structure per se, i.e. without the reactive polymer particles.

The “water-soluble electrophilic polymer carrying reactive electrophilic groups” that is employed in accordance with the present invention carries at least three reactive groups that are capable of reacting with amine groups in tissue and blood under the formation of a covalent bond. This water-soluble electrophilic polymer has a molecular weight of at least 1 kDa and a solubility in distilled water of 20° C. of at least 50 g/L.

The term “water absorption capacity” as used herein is a measure of the capability of the hemostatic patch to absorb water. The water absorption capacity is determined by weighing a sample of the dry sheet (weight=Wd) followed by immersion of the sample into distilled water (37° C.) for 45 minutes. Next, the sample is removed from the water and water clinging to the outside of the substrate is removed, following which the sample is weighed again (weight=Ww). The water absorption capacity=100%×(Ww−Wd)/Wd. The water adsorption capacity is indicative of the porosity of the substrate as well as of its ability to swell in the presence of water.

The term “collagen” as used herein refers the main structural protein in the extracellular space of various connective tissues in animal bodies. Collagen forms a characteristic triple helix of three polypeptide chains. Depending upon the degree of mineralization, collagen tissues may be either rigid (bone) or compliant (tendon) or have a gradient from rigid to compliant (cartilage). Unless indicated otherwise, the term “collagen” also encompasses modified collagens other than gelatin.

The term “gelatin” as used herein refers to a mixture of peptides and proteins produced by partial hydrolysis of collagen extracted from the skin, bones, and connective tissues of animals such as domesticated cattle, chicken, pigs, and fish. During hydrolysis, the natural molecular bonds between individual collagen strands are broken down into a form that rearranges more easily.

The term “polyoxazoline” as used herein refers to a poly(N-acylalkylenimine) or a poly(aroylalkylenimine) and is further referred to as POx. An example of POx is poly(2-ethyl-2-oxazoline). The term “polyoxazoline” also encompasses POx copolymers.

The present disclosure is related to systems, methods and devices for restoring hemostasis to a bleeding site of tissue, and in particular hemorrhage from an organ during minimally invasive procedures. Certain features, such as a fibrous carrier structure, can be designed to flexibly move through a trocar and be manipulated by robotic techniques at or near the bleeding site to stabilize the bleeding at the bleeding site. Certain feature of the hemostatic patch of this disclosure can allow the for easier delivery procedures, reduced time to delivery at the bleeding site, and reduce hemostasis.

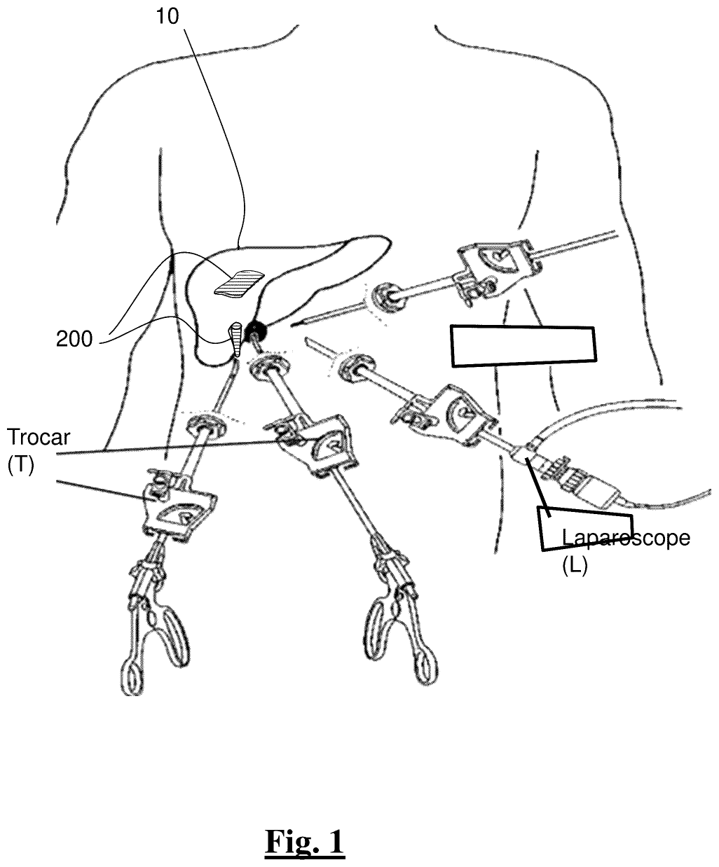

As an example, FIG. 1 depicts a schematic representation of a minimally invasive procedure with an example hemostatic device 200, also known as a hemostatic patch, delivered through a trocar and positioned at a bleeding site on an organ 10, in particular, the liver. Hemostatic device 200 can be designed to be delivered through a trocar to any internal bleeding site within the body, such as the liver, pancreas, spleen, stomach, gastrointestinal tract, kidney, bladder, reproductive organs, lungs, mediastinum, breast, lymph nodes, thymus, muscles, fat, heart, blood vessels, iliac arteries, carotid artery, vena cava, brain, and the like. Although not shown, hemostatic device 200 may also be designed for use during open surgery (e.g., not minimally invasive) and/or external use (e.g., on the skin of a subject).

Various studies disclosed herein compare device 200 (GATT-Patch; GATT Technologies BV, Nijmegen, The Netherlands) to those of comparative devices including Comparative Device 1 (TachoSil; Corza Medical, Westwood, MA, United States) and Comparative Device 2 (Surgifoam, Johnson and Johnson Ethicon, Bridgewater, NJ, United States), which is used in the studies described herein in combination with thrombin for more severe bleedings.

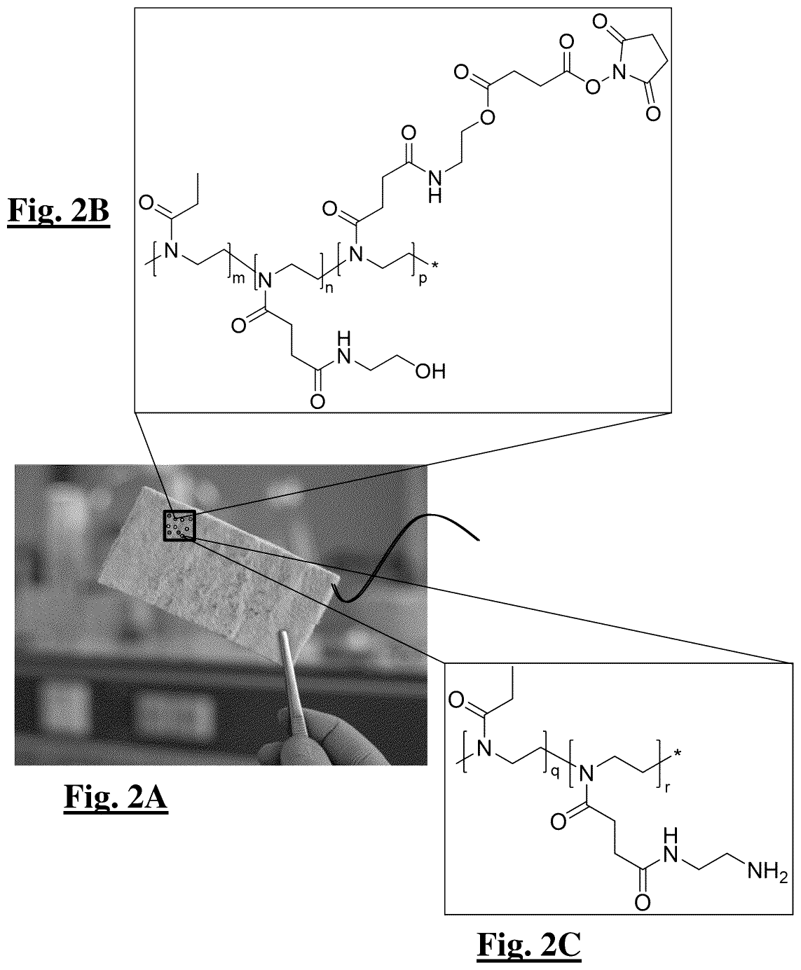

FIG. 2A depicts a pictorial representation of the hemostatic device 200. The Hemostatic Patch 200 according to the present invention can include a water-resistant cohesive fibrous carrier structure that holds small particles. The small particles include a distribution of a reactive electrophilic polymer depicted in FIG. 2B and polymer having nucleophiles depicted in FIG. 2C. The reactive electrophilic polymer is capable of covalently binding with host blood proteins and tissue as well as with reactive nucleophilic groups in the POx-polymer and in the fibrous carrier structure, and which thereby induces hemostasis and/or tissue-adhesion.

The reactive electrophilic polymer in FIG. 2B includes a functional NHS-ester side chain which can react (i) with amines at the target bleeding site (e.g., tissue, proteins, etc.,), (ii) with the crosslinking polymer of FIG. 2C, and (iii) the amines in the gelatin of the

Hemostatic Patch 200 to act as a hemostatic polymer. Crosslinking with tissue results in adhesion to tissue. Crosslinking to gelatin and the crosslinking polymer results in cohesion within the carrier. The backbone structure (which consists of tertiary amide groups) is generally stable under physiological conditions, but the presence of the ester group renders the side chains intrinsically biodegradable. This ester linkage also contributes to the degradability of the reactive electrophilic polymer. The reactive electrophilic polymer can include a composition of P(EtOx-OH-NHS) and forms a granulate embedded in the Hemostatic Patch 200. The backbone shown in FIG. 2B includes a copolymer having three species of monomer (terpolymer). In some examples, the P(EtOx-OH-NHS) can be formed with a 60-20-20 ratio (of about 60% POx, about 20% OH, and about 20% NHS). Of the 60%

Pox, the “Et” component can be inert and limit post polymerisation activation, whereas the “Me” components (40%) can be post polymerisation activated. The first monomer can have chain length m ranging from about 5 to about 5,000 repeating units. The second monomer can have a chain length n ranging from about 5 to about 5,000 repeating units. The third monomer can have a chain length p ranging from about 5 to about 5,000 repeating units. The PDI can range from between about 1.10 to about 2.50.

The crosslinking polymer of FIG. 2C includes amine side changes which can react with the electrophilic polymer of FIG. 2B to act as a crosslinking agent. The backbone structure includes tertiary amide groups and is generally stable under physiological conditions. The crosslinking polymer can include a composition of P(EtOx-NH₂). The backbone shown in FIG. 2C includes a copolymer having two species of monomer (bipolymer). In some examples, the P(EtOx-NH₂) can be formed with a 90-10 ration (of about 90% POx and about 10% NH₂). The first monomer can have chain length q ranging from about 5 to about 5,000 repeating units. The second monomer can have a chain length r ranging from about 5 to about 5,000 repeating units.

The molecular weight distribution or polydispersity index (PDI) of granulated reactive electrophilic polymer depicted in FIG. 2B and polymer crosslinks depicted in FIG. 2C is below 2.0 at release. Premature or excessive crosslinking between NHS-POx and NU-Pox and/or gelatin results in an increase of the PDI. If this crosslinking occurs, there will be less NHS groups readily available to which can adhere to the tissue and reduce the hemostatic control of Hemostatic Patch 200.

Hemostatic Patch 200 can restore hemostasis to a bleeding site by delivering, through a trocar T, the patch 200 near or about the bleeding site and positioning Hemostatic Patch 200 in contact with the tissue at the bleeding site. It is understood that Hemostatic Patch 200 could be used to restore hemostasis to the tissue within three minutes or less (e.g., 30 seconds) after putting Hemostatic Patch 200 in contact with the tissue. As applicable procedure guidelines change with respect to the use of hemostatic patch for treatment of laparoscopic L procedures, it is also conceivable that device 200 could be delivered via alternative techniques during minimally invasive procedures. Hemostatic Patch 200 can be understood as including features that are clearly described in Appendix A from U.S. Pat. Nos. 10,232,077 and 9,416,228, U.S. Publication No. 20220133949 (corresponding to U.S. Patent Application No. 17/573,564), US Publication No. 20220133948 (corresponding to U.S. Patent Application No. 17/573,541), U.S. Publication No. 20220133947 (corresponding to U.S. Patent Application No. 17/573,537); U.S. Publication No. 20220133943 (corresponding to U.S. Patent application Ser. No. 17/573,574), and U.S. Publication No. 20220153930 (corresponding to U.S. patent application Ser. No. 17/586,428), each of which are incorporated by reference in their entirety as if set forth verbatim herein.

The Hemostatic Patch 200 includes a water-resistant cohesive fibrous carrier structure that readily absorbs blood as blood can penetrate the interstitial space. The carrier is an absorbable non-woven porcine gelatin hemostat. It is made from 100% pharmaceutical grade gelatin and has a pH neutral character. It is used to stop capillary, venous, and arteriolar bleeding in general surgery. The material is multi-layered and can be tufted and peeled off in layers and applied gradually to achieve optimal results. This fibrous carrier structure can easily be impregnated with reactive polymer particles. Unlike impregnation with liquids, such dry impregnation does not affect the structural integrity or mechanical properties of the carrier structure. When blood is absorbed by the Hemostatic Patch 200, the reactive polymer particles within the sheet start dissolving as soon as they are ‘wetted’ by the blood, thereby allowing the electrophilic polymer to react with both reactive nucleophilic groups in the blood and tissue and reactive nucleophilic groups in the fibrous carrier structure, thereby inducing blood coagulation and tissue sealing, both of which contribute to hemostasis.

The blue colorant is added to the granulate that is impregnated into Hemostatic Patch 200 such that homogeneous distribution of granulate in the Hemostatic Patch 200 is easily visible and the surgeon is able to identify the patch when it is placed in direct contact with the wound. Hemostatic Patch 200 will contain blue colorant FD&C Blue No. 1, which is a certified color listed in 21 C.F.R. 82 and it has been approved for use in foods (21 C.F.R. 74.101), drugs (21 C.F.R. 74.1101) and cosmetics (21 C.F.R. 2101) and has also been used in other Class-III medical devices.

The reactive polymer particles may be homogeneously distributed within the interstitial space of the fibrous carrier structure in the sense that the particle density is essentially the same throughout the carrier structure. Alternatively, the reactive polymer particles may also be unevenly distributed throughout the carrier structure. For instance, if the hemostatic sheet is prepared in the form of a laminate of thin layers of fibrous carrier structure and layers of reactive polymer particles, the reactive polymer particle density within the sheet may fluctuate. For certain applications it may be advantageous if the reactive polymer particle density shows a gradient, e.g. in that the density of reactive particles is highest near the side of the sheet that is meant to applied onto a bleeding wound and lowest near the other side of the sheet.

The diameter distribution of the reactive polymer particles may suitably be determined by means of laser diffraction using a Malvern Mastersizer 2000 in combination with the Stainless-Steel Sample Dispersion Unit. The sample dispersion unit is filled with approx. 120 ml of cyclohexane, which is stabilized for 5 to 10 minutes at a stirring speed of 1800 rpm, followed by a background measurement (blank measurement). The sample tube is shaken and turned horizontally for 20 times. Next, about 50 mg is dispersed in the sample dispersion unit containing the cyclohexane. After the sample is introduced in the dispersion unit, the sample is stirred for one and a half minute at 1800 rpm to ensure that all particles are properly dispersed, before carrying out the measurement. No ultrasonic treatment is performed on the dispersed particles. Mean particle size is expressed as D [4,3], the volume weighted mean diameter (ΣnᵢDᵢ⁴)/(ΣnᵢDᵢ³).

In a particularly preferred embodiment, unlike the fibrous tissue sealant described in U.S. Pat. No. 8,545,871, the hemostatic patch of the present invention does not form a hydrogel until it is wetted upon delivery to the bleeding site, i.e. a water-swellable polymeric matrix that can absorb a substantial amount of water to form an elastic gel.

According to a particularly preferred embodiment, the Hemostatic Patch 200 is bioabsorbable, meaning that the carrier structure, the reactive polymer particles, and any other components of the Hemostatic Patch 200 are eventually absorbed in the body. Absorption of the carrier structure and reactive polymer particles typically requires chemical decomposition (e.g. hydrolysis) of polymers contained therein. Complete bioabsorption of the Hemostatic Patch 200 by the human body is typically achieved in approximately 1 to 10 weeks, preferably in approximately 4 to 6 weeks.

The Hemostatic Patch 200 typically has a non-compressed mean thickness of 0.5-25 mm. More preferably, the non-compressed mean thickness is in the range of 1-10 mm, most preferably in the range of 1.5-5 mm.

The dimensions of the Hemostatic Patch 200 preferably are such that the top and bottom of the sheet each have a surface area of at least 2 cm², more preferably of at least 10 cm² and most preferably of 25-50 cm². Typically, the sheet is rectangular in shape and has a length of 25-200 mm, and a width of 25-200 mm. Due to its flexibility, the Hemostatic Patch 200 of the present invention can suitably be applied to irregularly shaped bleeding sites. The haemostatic sheet may be applied layer on layer if an already applied sheet does not fully stop the bleeding.

Hemostatic Patch 200 can be cut into any suitable shape and size for the delivery method for laparoscopic procedure or for the size of the bleeding site. In addition, or alternatively thereto, Hemostatic Patch 200 can be shredded or otherwise formed into a construction similar to cotton candy due to the fibrous carrier structure. During a procedure, Hemostatic Patch 200 may be shredded or rolled into a ball and positioned within a cavity at the bleeding site, followed by placement of an unshredded patch over the bleeding site to restore hemostasis.

The Hemostatic Patch 200 preferably has a non-compressed density of less than 200 mg/cm³, more preferably of less than 150 mg/cm³ and most preferably of 10-100 mg/cm³ .

In one embodiment of the invention the reactive polymer particles are homogeneously distributed within the interstitial space of the fibrous carrier structure. In another embodiment of the invention the Hemostatic Patch 200 is a laminate comprising alternating layers of fibrous carrier structure and layers of the reactive polymer particles. In the latter embodiment, reactive polymer particles preferably have entered the layers of fibrous carrier structure that separate the layers of reactive polymer particles.

The Hemostatic Patch 200 preferably is essentially anhydrous. Typically, the Hemostatic Patch 200 has a water content of not more than 5 wt. %, more preferably of not more than 2 wt. % and most preferably of not more than 1 wt. %. The water absorption capacity of the Hemostatic Patch 200 preferably is at least 50%, more preferably lies in the range of 100% to 800%, most preferably in the range of 200% to 500%.

The Hemostatic Patch 200 of the present invention is preferably sterile.

The reactive polymer particles in the Hemostatic Patch 200 preferably include a water-soluble electrophilic polymer that carries reactive electrophilic groups selected from carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, olefins, glycidyl ethers, carboxyl, succinimidyl esters, sulfo succinimidyl esters, maleimido (maleimidyl), ethenesulfonyl, imido esters, aceto acetate, halo acetal, orthopyridyl disulfide, dihydroxy-phenyl derivatives, vinyl, acrylate, acrylamide, iodoacetamide and combinations thereof. More preferably, the reactive electrophilic groups are selected from carboxylic acid esters, sulfonate esters, phosphonate esters, pentafluorophenyl esters, p-nitrophenyl esters, p-nitrothiophenyl esters, acid halide groups, anhyinidrides, ketones, aldehydes, isocyanato, thioisocyanato, isocyano, epoxides, activated hydroxyl groups, glycidyl ethers, carboxyl, succinimidyl esters, sulfo succinimidyl esters, imido esters, dihydroxy-phenyl derivatives, and combinations thereof. Even more preferably, the reactive electrophilic groups are selected from halo acetals, orthopyridyl disulfide, maleimides, vinyl sulfone, dihydroxyphenyl derivatives, vinyl, acrylate, acrylamide, iodoacetamide, succinimidyl esters and combinations thereof. Most preferably, the reactive electrophilic groups are selected from maleimides, vinyl, acrylate, acrylamide, succinimidyl esters, sulfo succinimidyl esters and combinations thereof.

Suitable succinimidyl esters that may be employed include succinimidyl glutarate, succinimidyl propionate, succinimidyl succinamide, succinimidyl carbonate, disuccinimidyl suberate, bis(sulfosuccinimidyl) suberate, dithiobis(succinimidylpropionate), bis(2-succinimidooxycarbonyloxy) ethyl sulfone, 3,3′-dithiobis(sulfosuccinimidyl-propionate), succinimidyl carbamate, sulfosuccinimidyl(4-iodoacetyl)aminobenzoate, bis(sulfosuccinimidyl) suberate, sulfosuccinimidyl-4-(N-maleimidomethyl)-cyclohexane-1-carboxylate, dithiobis-sulfosuccinimidyl propionate, disulfo-succinimidyl tartarate; bis[2-(sulfo-succinimidyloxycarbonyloxyethylsulfone)], ethylene glycol bis(sulfosuccinimiclylsuccinate), dithiobis-(succinimidyl propionate), and combinations thereof.

Suitable dihydroxyphenyl derivatives that may be employed include dihydroxyphenylalanine, 3,4-dihydroxyphenylalanine (DOPA), dopamine, 3,4-dihydroxyhydroccinamic acid (DOHA), norepinephrine, epinephrine and catechol.

The use of a fibrous carrier structure in the Hemostatic Patch 200 offers the advantage that the reactive polymer particles can be homogeneously distributed throughout this carrier structure without difficulty. Such a homogeneous distribution is much more difficult to achieve in, for instance, foamed carrier structures.

The fibers in the fibrous carrier structure preferably have a mean diameter of 1-500 μm, more preferably of 2-300 μm and most preferably of 5-200 μm. The mean diameter of the fibers can suitably be determined using a microscope. Typically, at least 50 wt. %, more preferably at least 80 wt. % of the fibers in the fibrous carrier structure have a diameter of 1-300 μm and a length of at least 1 mm. Preferably, at least 50 wt. %, more preferably at least 80 wt. % of the fibers in the fibrous carrier structure have an aspect ratio (ratio of length to diameter) of at least 1000.

The fibrous carrier structure can include a felt structure, a woven structure, or a knitted structure. Most preferably, the fibrous carrier structure is a felt structure. Here the term “felt structure” refers to a structure that is produced by matting and pressing fibers together to form a cohesive material.

According to an embodiment, the fibrous carrier structure is biodegradable.

The nucleophilic polymer that is contained in the fibrous carrier structure may be homogenously distributed throughout fibers that are contained in the carrier's structure or it may be applied as an external coating layer. The presence of nucleophilic polymer in the carrier structure improves both adhesion and haemostatic properties of the Hemostatic Patch 200.

Preferably, the fibers of the fibrous carrier structure contain at least 5 wt. %, more preferably at least 10 wt. % and more preferably at least 50 wt. % of the nucleophilic polymer. Most preferably, the fibers consist of said nucleophilic polymer.

The nucleophilic polymer that is contained in fibers of the carrier structure typically contains at least 2 reactive nucleophilic groups, more preferably at least 5 reactive nucleophilic groups, even more preferably at least 10 reactive nucleophilic groups, most preferably at least 20 reactive nucleophilic groups. These reactive nucleophilic groups are preferably selected from amine groups, thiol groups, phosphine groups and combinations thereof. More preferably, these reactive nucleophilic groups are selected from amine groups, thiol groups and combinations thereof. Most preferably, the reactive nucleophilic groups are amine groups. These amine groups are preferably selected from primary amine groups, secondary amine groups and combinations thereof.

The nucleophilic polymer in the fibers of the fibrous carrier structure preferably has a nitrogen content of at least 1 wt. %, more preferably of 5-10 wt. % and most preferably of 15-25 wt. %.

The nucleophilic polymer is preferably selected from protein, chitosan, synthetic polymer carrying reactive nucleophilic groups, carbohydrate polymers carrying reactive nucleophilic groups and combinations thereof. More preferably, the nucleophilic polymer is selected from gelatin, collagen, chitosan and combinations thereof. Even more preferably, the nucleophilic polymer is gelatin, most preferably cross-linked gelatin.

Chitosan is a biodegradable, nontoxic, complex carbohydrate derivative of chitin (poly-N-acetyl-D-glucosamine), a naturally occurring substance. Chitosan is the deacetylated form of chitin. The chitosan applied in accordance with the present invention preferably has a degree of deacetylation of more than 70%.

The fibrous carrier structure preferably comprises at least 50 wt. %, more preferably at least 80 wt. % and most preferably at least 90 wt. % fibers containing a nucleophilic polymer carrying reactive nucleophilic groups.

In a preferred embodiment, the fibrous carrier structure comprises at least 50 wt. %, more preferably at least 80 wt. % and most preferably at least 90 wt. % fibers containing at least 50 wt. % of a nucleophilic polymer carrying reactive nucleophilic groups. According to a particularly preferred embodiment, the fibrous carrier structure comprises at least 50 wt. %, more preferably at least 80 wt. % and most preferably at least 90 wt. % fibers made from gelatin, collagen, or chitosan.

Preferred collagens do not possess telopeptide regions (“atelopeptide collagen”). The collagen employed in accordance with the present invention is preferably selected from the group of microfibrillar collagen, synthetic human collagen such as the type I collagen, type III collagen, or a combination of type I collagen and type III collagen. Collagen crosslinked using heat, radiation, or chemical agents such as glutaraldehyde may also be used.

In accordance with a preferred embodiment, the fibers in the fibrous carrier structure comprise at least 50 wt. %, more preferably at least 80 wt. % and most preferably at least 90 wt. % gelatin. The gelatin in the fibers preferably has a Bloom strength of 200 or more.

In a particularly advantageous embodiment, the fibrous carrier structure comprises at least 50 wt. %, more preferably at least 80 wt. % and most preferably at least 90 wt. % of partially cross-linked gelatin. The use of partially cross-linked gelatin offers the advantage that the fibrous carrier structure is both sufficiently stable and flexible at body temperature, and that swelling of the fibrous carrier structure does not result in the formation of a closed-pore fibrous gel structure.

In the preparation of the Hemostatic Patch 200, it can be advantageous to react a fraction of the reactive electrophilic groups in the electrophilic polymer of the of the reactive polymer particles with the reactive nucleophilic groups of the nucleophilic polymer. Thus, the reactive polymer particles may be fixated effectively within the fibrous carrier structure.

According to a preferred embodiment, the reactive nucleophilic groups of the nucleophilic polymer in the fibers of the fibrous carrier structure include amine groups and the reactive electrophilic groups of the electrophilic polymer in the reactive polymer particles are selected from carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, glycidyl ethers, carboxyl, succinimidyl esters, sulfosuccinimidyl esters,, imido esters, dihydroxy-phenyl derivatives, and combinations thereof.

According to another preferred embodiment, the reactive nucleophilic groups of nucleophilic polymers include thiol groups and the reactive electrophilic groups of the electrophilic polymer in the reactive polymer particles are selected from halo acetals, orthopyridyl disulfide, maleimides, vinyl sulfone, dihydroxyphenyl derivatives, vinyl, acrylate, acrylamide, iodoacetamide, succinimidyl esters, sulfosuccinmidyl esters and combinations thereof. More preferably, the reactive electrophilic groups are selected from succinimidyl esters, sulfosuccinimidyl esters, halo acetals, maleimides, or dihydroxyphenyl derivatives and combinations thereof. Most preferably, the reactive electrophilic groups are selected from maleimides or dihydroxyphenyl derivatives and combinations thereof.

In a preferred embodiment of the invention, the fibrous carrier structure does not comprise oxidised regenerated cellulose.

### Study Overview

This disclosure is more clearly understood with the corresponding studies discussed more particularly below with respect to laparoscopic procedures in hemorrhaging at different organs. It is understood that data is presented herein for purposes of illustration and should not be construed as limiting the scope of the disclosed technology in any way or excluding any alternative or additional embodiments.

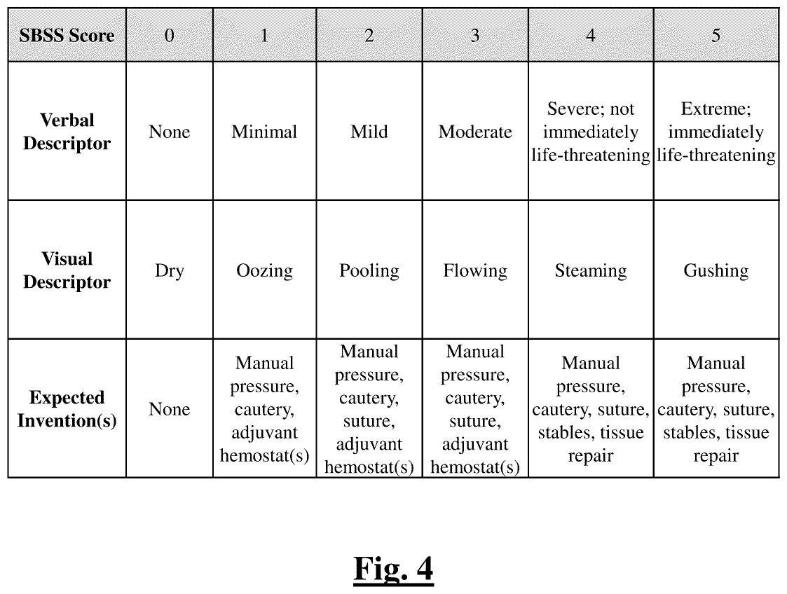

FIG. 4 shows a chart of surface bleeding severity scale (SBSS) scores verbal and visual descriptors and corresponding expected interventions. The SBSS is a validated bleeding scale developed for harmonized assessment of bleeding severity and evaluation of hemostasis. The SBSS consists of 6 subscales of bleeding (0=none, 1=minimal, 2=mild, 3=moderate, 4=severe; not immediately life-threatening, 5=extreme; immediately life threatening). The score has been successfully used to train surgeons in recognizing severities of bleeding related to in-and exclusion criteria in clinical investigations, and for assessment of endpoints of hemostasis for performance assessments. Hemostatic Patch 200 will be used as an adjunct to hemostasis for minimal, mild, moderate bleeding sites, defined by the Surface Bleeding Severity Scale (SBSS) 1-3.

The studies herein use the SBSS as the validated bleeding scale in preclinical evaluations of Hemostatic Patch 200 and in clinical investigations. Furthermore, as SBSS will be considered the predominant validated bleeding scale with which Hemostatic Patch 200 will be determined to be safe and performing as intended, the SBSS will be used in outward facing documents (e.g., Instructions for Use, Summary of Safety and Clinical Performance, etc.,) to delineate the clinical indication of Hemostatic Patch 200.

### Study 1: Non-GLP Study of Hemostatic Patch 200 versus Standard Suturing in Robotic Partial Nephrectomy

Hemostatic Patch 200 was studied as a solution for minimally invasive partial nephrectomy surgery. In general, partial nephrectomy surgeries are challenging to achieving hemostatic control with suturing. Here, Hemostatic Patch 200 presents (1) stronger hemostatic and adhesion properties than currently marketed products, (2) flexibility and pliability, and (3) ease of use that doesn't require manipulation of healthy kidney tissue. The present study evaluated the use of Hemostatic Patch 200 versus standard suturing as an acute hemostatic device during robotic partial nephrectomy surgery.

The surgery was performed by four consulting surgeons that routinely perform robotic kidney surgery, but with varying total experience. Four pigs were used during this terminal experiment. After anesthesia, using CO₂ influx in the abdominal cavity, four standard robotic ports of 8 mm or 12 mm (depending on the generation of Da Vinci robot) were introduced across the abdomen for the robotic arms and a standard fifth 12 mm port was placed for suction, introduction of sutures, introduction of Hemostatic Patch 200, etc. In each kidney, 4 reproducible lesions were created (e.g., 2 on the anterior and 2 on the posterior surface) with a robotic scissors. The size for the lesion was targeted to be circular with a diameter of 20 to 30 mm and a depth of 10 to 15 mm, with confirmed urine leakage from opening the calyx. At the time of creation of each lesion, the surgeon did not know whether Hemostatic Patch 200 or suturing would be the applied technique for hemostatic control. The sequence of Hemostatic Patch 200 and suturing on each kidney was done in a randomized fashion. Each kidney was treated n=2 by Hemostatic Patch 200 and n=2 by suturing. For the lesions treated with Hemostatic Patch 200, hemostatic control was performed by applying about half of a Hemostatic Patch 200 in the depth of the lesion with immediate topical application of about another half of a Hemostatic Patch 200, cut to a round shape with a diameter of about 3.5-5 cm, on top of the bleeding so that it overlapped non-bleeding tissue by at least 1 cm on all sides, according to the instructions for use (IFU). Thirty seconds of pressure with a saline-wetted gauze was initiated, after which hemostasis was checked by careful removal of the gauze. If bleeding persisted, an additional 30 seconds of pressure was applied. If bleeding persisted again, additional Hemostatic Patch 200 could be applied, and there was no maximum to how much Hemostatic Patch 200 could be used. For the lesions treated with standard suturing, hemostatic control was performed by inner-and outer renorrhaphy, e.g., suturing to close the kidney defect. The inner layer of suturing was performed in the depth of the lesion, each time clipping the suture to keep tension on the tissue and avoid sliding of the suture. After completion of the inner layer, early unclamping of the arteria renalis was performed to minimize the warm-ischemia time of the kidney. Subsequently, the outer layer of suturing was performed closing the more superficial defect of the kidney, in a similar fashion as the inner layer. If additional suturing was required for hemostatic control, this was allowed and there was no maximum number of sutures.

If there was active bleeding during hemostatic control, the severity of bleeding was adjudicated according to the Severity Bleeding Surface Scale (SBSS) (scale shown in FIG. 4) by two trained investigators that hold a certificate on the adjudication of bleeding on this scale of 0-5 bleeding severity: the following categorization was used: 0 represents no bleeding, 1 represents minimal bleeding, 2 represents mild bleeding, 3 represents moderate bleeding, 4 represents severe bleeding, and 5 represent extreme, life-threatening bleeding.

After all lesions were created and hemostatic control was achieved, the ureter was clamped to initiate a hydronephrotic state of the kidney and increase the pressure and likelihood of urine leakage from the lesions. At the time of necropsy, which was at least 3 hours after the clip on the ureter has been placed, urine leakage was tested by performing a burst pressure test with a catheter placed in the ureter just proximal of the previously placed clip. The catheter was connected to a syringe containing saline mixed with methylene blue to increase pressure until blue liquid was visible at kidney lesions. Pressure was monitored with a digital pressure monitor (GMH 3151, Geisinger, Regenstauf, Germany) connected to a digital pressure sensor (GMSD 25 MR-K31, Greisinger, Regensatauf, Germany).

During surgery, an excel spreadsheet with an automatic time capture function (hours: min:sec) was used to determine the time at which critical steps of the procedure were performed. Based on these timepoints, several surgery times were calculated:

Arterial clamping time, defined as the time that a Bulldog clamp was placed on the arteria renalis for the creation of the lesion and (partly) hemostatic control.

Hemostatic control, defined for the Hemostatic Patch 200 group as the time between the introduction of a gauze or Hemostatic Patch 200 until removal of the gauze after Hemostatic Patch 200 application, and for the suturing group as the time between the start of suturing until removal of the needles once suturing was completed.
Active bleeding, defined as the time that there was active bleeding during hemostatic control.
Total procedure time, defined by the start of the procedure triggered by placing the Bulldog clamp on the arteria renalis, the creation of the lesion, and the hemostatic control period until hemostatic control was achieved.

For the hemostatic performance of Hemostatic Patch 200 as compared with the standard suture technique, the following two endpoints were established to evaluate acute and persistent hemostasis:
Initial hemostasis, defined as an SBSS of 0 at the end of the hemostatic control period.
Rebleeding after at least 2 hours, defined as an SBSS>0 at the robotic relook.

During partial nephrectomy, the calyx of the kidney may be opened and for the assessment of whether Hemostatic Patch 200 versus suturing offers a solution in urine sealing, this was evaluated at three different timepoints during the study:
Initial passive urine leakage, as defined by no evidence of urine leakage at the end of the hemostatic control period.
Hydronephrotic urine leakage after the clip on the ureter has been in place for at least 2 hours.
Burst pressure of urine sealing, at least 3 hours after the clip was put on the ureter.

Lastly, while the usability of Hemostatic Patch 200 has been demonstrated in open surgical procedures, it is critical to collect information on the use of Hemostatic Patch 200 during minimal invasive surgery, in this case being robotic. During the use of Hemostatic Patch 200, the following information was therefore collected:
The ease and effectiveness of introducing Hemostatic Patch 200 through a minimally invasive trocar.
Maneuvering of Hemostatic Patch 200 in the body and placing it onto the bleeding site with robotic tools.
Applying pressure with a wet gauze to achieve adequate adhesion of Hemostatic Patch 200 to the tissue and achieving hemostasis.
Adhesion of Hemostatic Patch 200 to the tissue at the time of necropsy, assessed by attempting to remove Hemostatic Patch 200 using a forceps on the edge of the patch, rates as “pass” if the patch could not easily be removed.

A total of 32 lesions were created, treated with Hemostatic Patch 200 (n=16) and standard suturing (n=16). Mean arterial blood pressure at the of creation of the lesion was comparable between the Hemostatic Patch 200 and suturing groups: 68.3±12.5 mmHg versus 71.1±12.3 mmHg (P=0.53). The size of the lesion, as confirmed at the time of necropsy, was also comparable for the diameter (Hemostatic Patch 200 2.6±0.4 cm versus suturing 2.6±0.5 cm; P=0.85) and depth (Hemostatic Patch 200 1.4±0.1 cm versus suturing 1.4±0.1 cm; P=0.30).

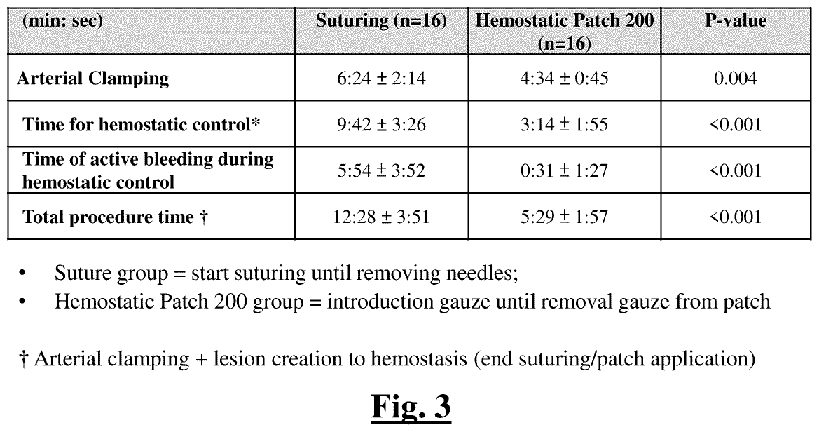

FIG. 3 shows a table summarizing procedural times of Hemostatic Patch 200 versus suturing techniques. As demonstrated, Hemostatic Patch 200 reduced all specific times significantly. Specifically, Hemostatic Patch 200 reduced the arterial clamping time by 40% (4:34±0:45 for Hemostatic Patch 200 versus 6:24±2:14 for suturing; P=0.004), the time to achieve hemostatic control by 300% (3:14±1:55 for Hemostatic Patch 200 versus 9:42±3:26 for suturing; P<0.001), the active bleeding time by 1142% (0:31±1:27 for Hemostatic Patch 200 versus 5:54±3:52 for suturing; P<0.001) and the total procedure time by 227% (5:29±1:57 for Hemostatic Patch 200 versus 12:28±3:51 for suturing; P<0.001).

Active bleeding during hemostatic control was 19% with Hemostatic Patch 200 versus 94% with suturing (P<0.001), but initial hemostasis was achieved in 100% with both Hemostatic Patch 200 and suturing (P>0.99).

Hemostatic Patch 200 achieved hemostasis with 30 seconds of pressure in 81% (N= 13/16), with about half a Hemostatic Patch 200 put in the depth of the lesion and about half a Hemostatic Patch 200 on top. In three cases, additional hemostatic maneuvers were required because of bleeding after 30 seconds of pressure:
Case 1: Two times an additional 30 seconds of pressure was needed to achieve hemostasis. Active bleeding occurred only for several seconds with an SBSS 1 (e.g., minimal) during resolution. The time to hemostasis from initiating pressure on the first Hemostatic Patch 200 to hemostasis was 02:28 min.
Case 2: An additional 30 seconds of pressure was applied, after which bleeding continued. An additional half a Hemostatic Patch 200 was applied on top of the previous Hemostatic Patch 200 after which bleeding still continued. Finally, a third half a Hemostatic Patch 200 partly on top of the previous hemostatic patches 200 was applied to achieve hemostasis. Active bleeding time during resolution was 05:00 min, with an SBSS of 2-3 (e.g., mild to moderate bleeding). The time to hemostasis from initiating pressure on the first Hemostatic Patch 200 to hemostasis was 07:32 min.
Case 3: An additional 30 seconds of pressure was applied, after which bleeding continued from out of the depth of the lesion. Subsequently, an additional half a Hemostatic Patch 200 was added in the lesion and another half a Hemostatic Patch 200 on top. Bleeding still continued, and finally the addition of a fourth half a Hemostatic Patch 200 on top of the previous hemostatic patches 200 resulted in hemostasis. Active bleeding time during resolution was 03:20 min, with an SBSS of 2 (e.g., mild bleeding). The time to hemostasis from initiating pressure on the first patch 200 to hemostasis was 06:42 min.

Suturing also resulted in hemostasis, but in 94% with active bleeding during hemostatic control. Active bleeding was typically of the severity of SBSS 1-2 (e.g., minimal and mild), with the following exceptions:
Unclamping of the renal artery resulted in an acute pulsating bleeding with an SBSS 5 (e.g., life-threatening) for 05:45 min. It was resolved by additional suturing, after which an SBSS of 1 persisted for another minute.
Unclamping of the renal artery resulted in an acute pulsating bleeding with an SBSS 4-5 (e.g., severe to life-threatening) for 02:29 min. It was resolved by additional suturing and bleeding stopped.
During suturing, an SBSS of 2-3 (e.g., mild and moderate) occurred for 10:16 min before hemostatic control was achieved.

The time between achieved hemostasis and relook was 3:50±1:14 hrs for Hemostatic Patch 200 versus 3:49±1:08 hrs for suturing (P=0.96). Rebleeding at robotic relook occurred in 0% for Hemostatic Patch 200 versus 12.5% (n= 2/16) for suturing and consisted of an SBSS 1 (e.g., minimal bleeding) (P=0.48). The time between the clip on the ureter and robotic relook was 3:28±1:08 hrs for Hemostatic Patch 200 versus 3:28±1:09 hrs for suturing (P=0.99). There was no urine leakage upon the relook in any of the groups. At the time of necropsy, burst pressure measurements were performed in 3 out of the 4 pigs. Unfortunately, the pressure system malfunctioned during the assessment in the first pig and the pressures could not be read accurately, after which a different pressure system was used in the consecutive pigs, with the following values:

Pig 2:
First kidney: all n=2 Hemostatic Patch 200 and n=2 sutures held a 143 mmHg pressure. There was no confirmed burst pressure for these n=2 Hemostatic Patch 200 and n=2 suturing lesions.
Second kidney: n=1 Hemostatic Patch 200 burst at 31 mmHg, n=1 suturing burst at 91 mmHg, and the remaining n=1 Hemostatic Patch 200 and n=1 suturing held a 138 mmHg pressure. There was no confirmed burst pressure for these 2 lesions.

Pig 3:

First kidney: n=1 Hemostatic Patch 200 burst at 84 mmHg, n=1 suturing burst at 143 mmHg, and the other remaining n=1 Hemostatic Patch 200 and n=1 suturing held 178 mmHg pressure. There was no confirmed burst pressure for these 2 lesions.
Second kidney: n=1 suturing burst at 75 mmHg, n=1 suturing burst at 100 mmHg, n=1 Hemostatic Patch 200 burst at 108 mmHg, and n=1 Hemostatic Patch 200 burst at 200 mmHg.

Pig 4:
First kidney: n=1 suturing burst at 63 mmHg, n=1 Hemostatic Patch 200 burst at 63 mmHg, n=1 suturing burst at 174 mmHg, and n=1 Hemostatic Patch 200 held a 262 mmHg pressure without confirmed burst pressure.
Second kidney: n=1 Hemostatic Patch 200 burst at 210 mmHg; both n=2 suturing burst at 238 mmHg, and n=1 Hemostatic Patch 200 burst at 253 mmHg.

Considering that pressures in the urinary collection system remain low in clinical situations, the burst pressures of all Hemostatic Patch 200 and suture lesions confirm the clinical safety of using these techniques to close deep partial nephrectomies.

At the time of necropsy, adhesion of Hemostatic Patch 200 to the tissue was rates as “pass” for all applications. Hemostatic Patch 200 could not easily be removed by pulling on the edge of the patch.

During surgery and the robotic relook, kidney damage as a result of suturing the lesions was observed. To suture a kidney defect, approximation of tissue is required for hemostasis, and suturing therefore requires pulling healthy tissue towards each other. Suturing this healthy tissue results in closing of blood vessels in the tissue, intraparenchymal hematoma, tension, and other factors that may prevent wound healing. This phenomenon as a result of suturing is also well-known. Importantly, this did not occur with Hemostatic Patch 200 as there is no need to manipulate the healthy tissue.

Sixteen lesions treated with Hemostatic Patch 200, of which 14 lesions were treated with the initial two half hemostatic patches 200 (˜5×˜5 cm; one in the depth of the lesion and one superficial), 1 lesion required 4 half hemostatic patches 200 for treatment, and 1 lesion required 5 half hemostatic patches 200 for treatment. Therefore, a total of 37 half hemostatic patches 200 were introduced, with the following findings:

1. Hemostatic Patch 200 was easily rolled up without damaging the patch.

2. Hemostatic Patch 200 was easily introduced through the trocar without damaging the patch; only in one case was there damage of the patch, but the patch could still be used for hemostasis.

3. In none of the cases did Hemostatic Patch 200 stick to the trocar or robotic instruments so that it could no longer be used.

4. In all cases, Hemostatic Patch 200 could be easy maneuvering in the body and placed onto the bleeding site.

5. Pressure with a wet gauze during application (as per IFU) was easily applied using the multiple robotic arms, and was considered to be adequate based on adhesion testing at necropsy confirming that after more than 3 hours, Hemostatic Patch 200 adhesion to the kidney tissue was strong.

6. After the wet gauze application, the gauze did not stick to Hemostatic Patch 200 and could easily be removed without damaging the patch or pulling off the patch.

In summary, Hemostatic Patch 200, as compared with the standard of care of closing partial nephrectomy lesions with suturing, presents a solution that is quicker in all different aspects of the procedure, reduces blood loss during surgery, and reduces damage to the (healthy) kidney.

More specifically, and important for the evidence supporting use of Hemostatic Patch 200 in a broad clinical setting:

1. Hemostatic Patch 200 was reliable in achieving hemostasis within 30 seconds of pressure, per IFU, in 81% of these cases. Even if hemostasis was not immediately achieved in 19% of the cases, additional pressure on Hemostatic Patch 200 or the addition of an additional layer of Hemostatic Patch 200 are solutions to achieve hemostasis without the need for further surgical complexity or the use of other devices/agents. Therefore, and particularly when compared to the complex practice of robotic suturing, it may even reduce the learning curve of robotic surgery.

2. The present study provides strong evidence that Hemostatic Patch 200 can be used during minimally invasive surgery, as it could be introduced through a trocar easily, could be placed on the wound without it being compromised by other tissue or the surgical tools, and pressure could be applied appropriately for Hemostatic Patch 200 to adhere to the tissue and initiate acute and persistent hemostasis without rebleeding.

Hemostatic Patch 200 demonstrated to have exceptional hemostatic performance in kidney surgery, as well as being resistant to urine. Hemostatic Patch 200 could seal off the calyx to allow urine sealing, even up to bursting pressures considerably higher than what is required in clinical practice. This provides important evidence on the broad application of Hemostatic Patch 200.

### Study 2: Study of Hemostatic Patch 200 versus Comparative Device 1 in Porcine Robotic Liver Bleeding Model

Four pigs of Rattlerow Seghers origin and 60-80 kg weight were used during this terminal experiment. Animals were sedated using ketamine (10 mg/kg), medetomidine (40 ug/kg) and morphine (0.1 mg/kg), and subsequently anaesthetized with propofol (1-3 mg/kg). Intubation was performed after Lidocaine spray (10% spray) on the trachea.

Anesthesia was intraoperatively and the animals further received Ringer lactate solution (10 ml/kg/u). A mean arterial blood pressure higher than 60 mmHg was maintained using L-noradrenaline, if required. Animals were monitored for and by an electrocardiogram, pulse oximetry, capnography and spirometry, invasive blood pressure, and temperature. At the end of the experiment, animals were euthanized with a lethal dose of pentobarbital (50 mg/kg) or T61.

Animals were placed in supine position on the operating table in front of a Da Vinci robot. After anesthesia, using CO₂ influx in the abdominal cavity, four standard robotic ports of 12 mm were introduced across the abdomen for the robotic arms and one standard 12 mm port was furthermore placed for suction, introduction of sutures, introduction of Hemostatic Patch 200, etc. The intra-abdominal pressure was set at 8 mm Hg during the entire procedure.

Lesions of the liver parenchyma and the main vessels of the liver were created by using sharp non-cauterizing scissors, sharp bipolar-cauterizing scissors, or bipolar vessel sealing system. A series of 8-10 lesions were aimed per animal.

After lesion creation, the severity of bleeding was adjudicated according to the Severity Bleeding Surface Scale (SBSS) by consensus of two trained investigators that hold a certificate on the adjudication of bleeding on this scale of 0-5 bleeding severity: 0 represents no bleeding, 1 represents minimal bleeding, 2 represents mild bleeding, 3 represents moderate bleeding, 4 represents severe bleeding, and 5 represent extreme, life-threatening bleeding. Hemostasis was considered to be an SBSS of 0.

Per protocol, half of lesions were treated with hemostatic patch. Before introduction in the trocar, Hemostatic Patch 200 was cut to the appropriate size so that it overlapped non-bleeding tissue by at least 1 cm on all sides. Hemostatic Patch 200 was subsequently rolled over its long axis and grasped at the most distal tip to be introduced through a 12 mm size trocar. After navigation to the bleeding and unrolling, Hemostatic Patch 200 was positioned on the bleeding and applied by 30 seconds of pressure with a saline-wetted gauze, per IFU, after which hemostasis was checked by careful removal of the gauze. If hemostasis was not yet achieved, either additional pressure, or additional (pieces of) Hemostatic Patch 200 were applied. Hemostasis was checked at predetermined times of 30 seconds, 1 minute, 3 minutes and the endpoint of 5 minutes. The time to hemostasis was considered to be the time that hemostasis was achieved and maintained.

The other half of the lesions were treated with Comparative Device 1. Comparative Device 1 was cut to the appropriate size so that it overlapped non-bleeding tissue by at least 1 cm on all sides. It was subsequently folded in a zigzag way after which it was grasped at the most distal tip and introduced through a 12 mm trocar. After unfolding, it was navigated to the bleeding. After positioning on the bleeding, it was applied by 3 minutes of pressure with a saline-wetted gauze. Hemostasis was checked at 3 minutes. If hemostasis was not yet achieved, either additional pressure, or additional (pieces of) Comparative Device 1 were applied.

The primary outcome was hemostatic performance as determined by the time-to hemostasis, with a pass or fail at the endpoint of 5 minutes. Furthermore, the usability of Hemostatic Patch 200 and Comparative Device 1, in terms of preparation for introduction through a trocar, the introduction through a trocar, the navigation to the bleeding site, the unrolling/unfolding and positioning on the bleeding site, the application of the patch with a wet gauze, and the removal of the gauze after application, were all scored as “pass” if successful without rendering the patch to be unusable, or “fail” if the patch could no longer be used.

A total of 36 lesions were created, which were treated with Hemostatic Patch 200 (n=18) and Comparative Device 1 (n=18): n=16 superficial lesions (n=8 Hemostatic Patch 200, n=8 Comparative Device 1), n=8 partial liver resections (n=4 Hemostatic Patch 200, n=4 Comparative Device 1) and n=12 deep metastasectomy (n=6 Hemostatic Patch 200, n=6 Comparative Device 1).

### Superficial Lesions

A superficial lesion was created with sharp scissors. The size of the lesions were approximately 1×1 cm with a depth of about 0.3-0.5 cm. The bleeding severity was noted. A piece of Hemostatic Patch 200 or Comparative Device 1, about 4×4 cm for both, was introduced through the trocar and placed onto the lesion. After pressure with a saline-wetted gauze over the surface of the patch for 30 seconds for Hemostatic Patch 200 and 3 minutes for Comparative Device 1, the gauze was removed and hemostasis was checked. If no hemostasis was achieved, additional pressure was applied.

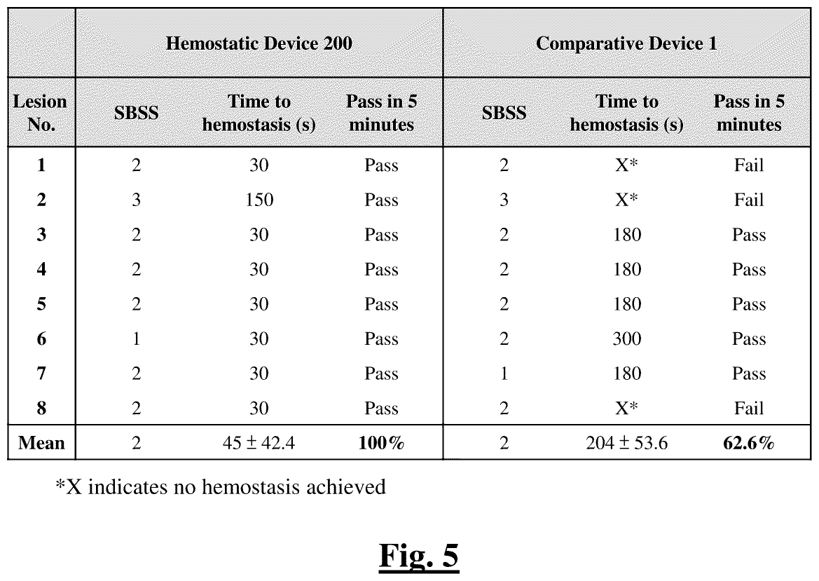

FIG. 5 provides a summary of the superficial lesions; the distribution of SBSS was equal between the Hemostatic Patch 200 and Comparative Device 1 treated lesions with the mean SBSS being 2.

With Hemostatic Patch 200, in 7 out of 8 cases (88%), hemostasis was achieved within 30 seconds of pressure. In one case additional pressure was needed and hemostasis was achieved in 150 seconds. All applications of Hemostatic Patch 200 reached persistent hemostasis within the endpoint of 5 minutes, with a mean time to hemostasis of 45±42 seconds.

With Comparative Device 1, in 4 out of 8 cases (50%), hemostasis was achieved after the initial 3 minutes (180 seconds) of pressure with the wet gauze. One lesion achieved hemostasis in 5 minutes (300 seconds) after additional pressure was provided. A total of 5 cases achieved persistent homeostasis within the endpoint of 5 minutes with a mean time to hemostasis of 204±54 seconds. Three lesions (38%) did not reached hemostasis within 5 minutes.

### Partial Liver Resection

A partial liver resection was created with sharp scissors. The size of the lesions were approximately 5-7 cm long and a thickness of about 1-1.5 cm. The bleeding severity was noted. A full Hemostatic Patch 200 or Comparative Device 1 was introduced through the trocar and placed on the lesion. Pressure was given on the surface of the patch with a saline-wetted gauze for 30 seconds for Hemostatic Patch 200 and 3 minutes for Comparative Device 1, after which the gauze was removed and hemostasis was checked. If no hemostasis was achieved, additional pressure was applied.

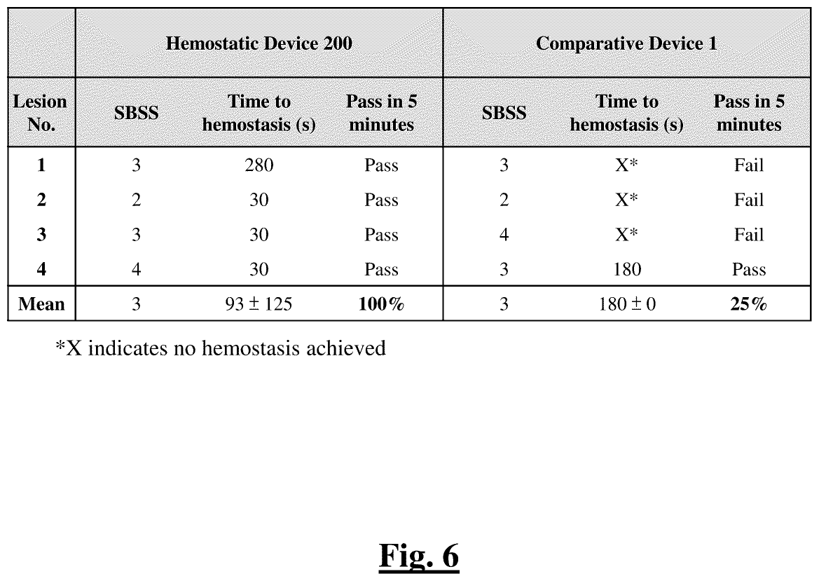

FIG. 6 provides a summary of the partial liver resections; the distribution of SBSS was equal between the Hemostatic Patch 200 and Comparative Device 1 treated lesions, with a mean SBSS of 3.

With Hemostatic Patch 200, in 3 out of 4 cases (75%), hemostasis was achieved within 30 seconds of pressure. In one case, additional pressure was required and hemostasis was achieved in 280 seconds. All applications of Hemostatic Patch 200 reached persistent hemostasis within the endpoint of 5 minutes, with a mean time to hemostasis of 93±125 seconds.

With Comparative Device 1, 1 out of 4 cases (25%) reached hemostasis after the initial 3 minutes (180 seconds) of pressure with the wet gauze. The other 3 lesions failed to reach hemostasis within 5 minutes.

### Deep Metastasectomy

A deep metastasectomy was created with a vessel sealer. The size of the lesions were approximately 2.5-3×2.5-3 cm with a depth of about 2-2.5 cm. The bleeding severity was noted.

For Hemostatic Patch 200, a piece of Hemostatic Patch 200 was introduced through the trocar and placed into the depth of the lesion, with a second piece introduced and placed on top of the lesion.

For Comparative Device 1, a piece was introduced through the trocar and placed directly on the lesion, per instructions for use. Pressure was given on the surface of the patch with a saline wetted gauze for 30 seconds for Hemostatic Patch 200 and 3 minutes for Comparative Device 1, after which the gauze was removed and hemostasis was checked. If no hemostasis was achieved, additional pressure was applied.

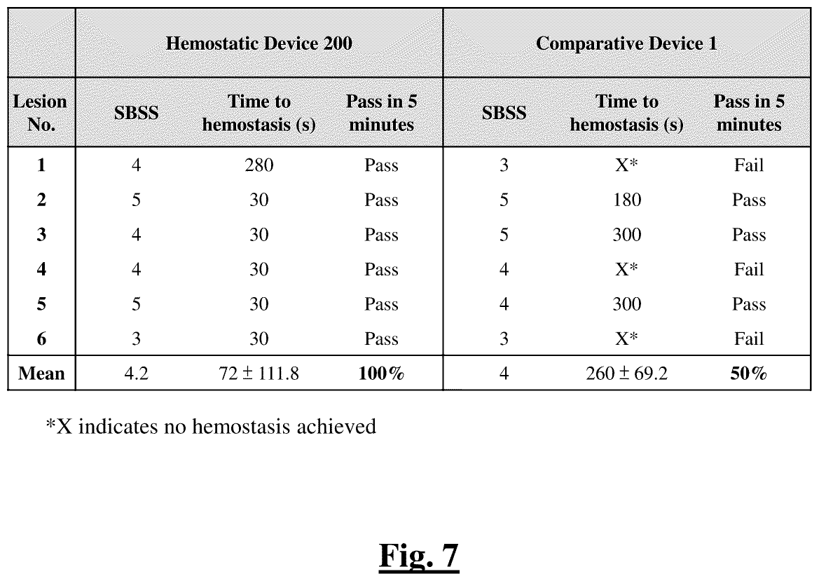

FIG. 7 provides a summary of the deep metastasectomy; the distribution of SBSS was equal between the Hemostatic Patch 200 and Comparative Device 1 treated lesions, with a mean SBSS of 4.2.

With Hemostatic Patch 200, in 5 out of 6 cases (83%), hemostasis was achieved within 30 seconds of pressure. In one case (the first case), one layer of Hemostatic Patch 200 was applied, which got stuck to the gauze and broke when removing the wet gauze. The bleeding was managed by placing a part of the patch into the depth of the lesion and subsequently placing a second part of a Hemostatic Patch 200 on top of that. This resulted in a total time of hemostasis of 280 seconds. This method of using Hemostatic Patch 200 was used for the remainder of the cases and resulted in hemostasis at 30 seconds in all those cases. All applications of Hemostatic Patch 200 reached persistent hemostasis within 5 minutes with a mean time to hemostasis of 72±112 seconds.

With Comparative Device 1, one out of 6 cases (17%) reached hemostasis after the initial 3 minutes (180 seconds) of pressure with the wet gauze. In two cases, hemostasis was reached in 5 minutes after additional pressure. A total of 3 lesions reached hemostasis within the endpoint of 5 minutes with a mean time to hemostasis of 260±69 seconds. The remaining 3 lesions (50%) did not reach hemostasis within 5 minutes.

### Minimally Invasive Usability

There were a total of 25 uses of (parts of) Hemostatic Patch 200. In nearly all cases requiring different sizes of Hemostatic Patch 200 ranging from about 4×4 cm to a full Hemostatic Patch 200 of 10×5 cm, the patch could be rolled over its long axis easily and introduced through a 12 mm trocar without damage to the patch; only once did the patch get stuck in the trocar, but was still usable after being pushed through the trocar. The trocar was subsequently still useable without difficulties. In all cases, after introduction, Hemostatic Patch 200 could be navigated to the bleeding site without rendering it unusable because of adhering to other structures or to the surgical instruments. It could be unrolled and positioned on the bleeding site and subsequent pressure with a wet gauze caused the patch to be sufficiently adherent to the tissue, thereby causing hemostasis to occur quickly after 30 seconds of pressure in the majority of cases, and maintaining its hemostatic performance through further timepoints. With Comparative Device 1, twice the patch broke during introduction through the trocar; in both instances the patch was still usable. In 6 out of 18 cases, the surgeon reported more difficulty to unfold the Comparative Device 1 in reference to Hemostatic Patch 200, and navigating it to the bleeding site and apply it to the bleeding.

A total of 36 lesions were created in four pigs, of which 18 lesions were treated with Hemostatic Patch 200 deep metastasectomy lesions, Hemostatic Patch 200 achieved hemostasis in 18 out of 18 cases (100%) within the endpoint of 5 minutes with a mean time to hemostasis of 64±83 seconds, while Comparative Device 1 achieved hemostasis in only 9 out of 18 (50%) lesions within the endpoint of 5 minutes and with a mean time to hemostasis of 220±60 seconds (for those achieving hemostasis). Moreover, Hemostatic Patch 200 achieved hemostasis after 30 seconds of pressure in 15 out of 18 lesions (83%).

These data provide evidence that hemostasis of up to life-threatening bleeding during robotic liver resection can be achieved quickly and reliably with Hemostatic Patch 200, particularly when compared to the standard of care product, Comparative Device 1, which is approved for minimally invasive surgery in Europe. In addition to the hemostatic performance, Hemostatic Patch 200 its lack of extensive preparation and ease-of-use, even in minimally invasive settings, may help reduce the need for conversion to open surgery to control bleedings, and thereby significantly benefit patient outcomes.

This data provides evidence on several important aspects of Hemostatic Patch 200:

1. The usability of Hemostatic Patch 200 is confirmed in this study with 25 introductions and uses of Hemostatic Patch 200 ranging from a 4×4 size to its full size of 10×5 cm. When rolled over its long axis, it could be easily introduced without damaging the patch through a trocar size of 12 mm. In only one case did the patch get stuck in the trocar, even though it could still be used then. Furthermore, in all cases, Hemostatic Patch 200 was easily navigated to the bleeding and positioned onto the bleeding with adequate pressure upon application. This is also reflected in the usability questionnaire completed by the independent surgeon that performed the procedure. The surgeon noted that use of Comparative Device 1 was more difficult, despite the surgeon being an expert in use of Comparative Device 1 on a weekly basis.

2. Hemostatic Patch 200 could be used during a variety of bleeding scenarios, representing also challenging bleedings requiring a flexible and pliable product for adequate positioning of the bleeding site.

3. Even in challenging minimally invasive settings, the 30-seconds application time of Hemostatic Patch 200 is adequate in achieving hemostasis in the vast majority of cases of soft tissue (e.g. in this case liver) bleeding (e.g. 83%), similar to what is achieved in other open and minimally invasive preclinical and (open) clinical studies.

4. While the initial indication for Hemostatic Patch 200 is proposed to be for use on minimal, mild, and moderate bleeding, this study supports use of Hemostatic Patch 200 in severe bleedings for potential future regulatory approvals.

### Study 3: GLP Evaluation of Efficacy and Safety of the Hemostatic Patch 200 Following Functional Laparoscopic Application of Bleeding Sites in a Swine Model

To assess the use of Hemostatic Patch 200 in minimally invasive surgery (MIS), a GLP preclinical study was undertaken to evaluate the effectiveness and safety of Hemostatic Patch 200 when applied in a laparoscopic setting. This study used similar methods to the previous open GLP study, with the aim of being to demonstrate that Hemostatic Patch 200 performs as intended in a laparoscopic setting by assessing hemostatic performance, rebleeding rates, safety, and degradation, and comparing these with the open GLP study results.

Four animals were fasted, anesthetized and prepared for a chronic surgical procedure; 3 animals were treated with Hemostatic Patch 200 and one animal with a positive control of Comparative Device 2+Thrombin. The control of Comparative Device 2+Thrombin was chosen based on the same intended use and similar product design and being used in the previous open GLP study.

The protocol for the GLP laparoscopic study was written to resemble the previously performed GLP study on Hemostatic Patch 200 in open surgery as much as possible, with the obvious difference being the approach of laparoscopic vs. open surgery. There are a few distinct other differences:

1. The open GLP study included 2 control arms with Comparative Device 2+Thrombin and a third comparative device (Hemopatch, Baxter International Inc., Deerfield, IL, United States), and the laparoscopic GLP study only included a single control arm of Comparative Device 2+Thrombin. Because Hemostatic Patch 200 already demonstrated to be safe and effective in the previous open GLP study as compared to both controls, it was deemed unnecessary to again include 2 control arms and for the sake of 3Rs, the number of animals were reduced by including only a single control arm.

2. The open GLP study included 3 survival cohorts, and the laparoscopic study only included a single cohort. Because in the open GLP study Hemostatic Patch 200 already demonstrated degradation of the patch within 4 weeks, an 8-week endpoint was deemed unnecessary. Hemostatic Patch 200 also showed appropriate local tissue response during the course of degradation based on ISO 10993-6. A laparoscopic relook at 3 days to check for rebleeding was performed instead of the open GLP 72 hrs sacrifice in order to not have to include a separate survival cohort for the particular endpoint of rebleeding. Thereby, for the sake of 3Rs, the number of animals was reduced.

3. For the endpoint of time-to-hemostasis, the application time of Hemostatic Patch 200 in the open GLP study was 1 minute, while in the laparoscopic GLP study it was 30 seconds, per the instructions for use (IFU) of Hemostatic Patch 200. All other observation times (e.g., 1, 3, and 5 minutes) were similar to the open GLP study. The 30-seconds application time is justified by further (pre)clinical evidence on Hemostatic Patch 200 demonstrating that ˜>80% of bleedings are hemostatic after 30 seconds application. In addition, in an acute laparoscopic non-GLP study, 16 biopsy punch bleedings similar as in the GLP study were treated with Hemostatic Patch 200 in 2 pigs, and n= 13/16, 81%, reached hemostasis at 30 seconds with the additional n= 3/16, 19%, reaching hemostasis at 60, 60, and 80 seconds after an additional 30 seconds of pressure.

Besides these differences, the bleeding model was identical where 8 lesions of minimal, mild or moderate bleeding were created on the liver with an 8 mm biopsy punch, treated by a 2.5 by 2.5 cm piece of Hemostatic Patch 200, and included in the present study. Endpoints of rebleeding, hematoma formation, product migration, adhesion formation, product degradation, clinical adverse events, clinical pathology, and histopathology were comparable, so that the evaluation of Hemostatic Patch 200 versus Comparative Device 2+Thrombin in the laparoscopic GLP study could be compared against the historical results of the open GLP study.

Audited data sheets of all the surgical procedures and the endpoints evaluated during the procedures were used for a preliminary assessment of safety and performance of Hemostatic Patch 200 in a laparoscopic setting, as discussed supra.

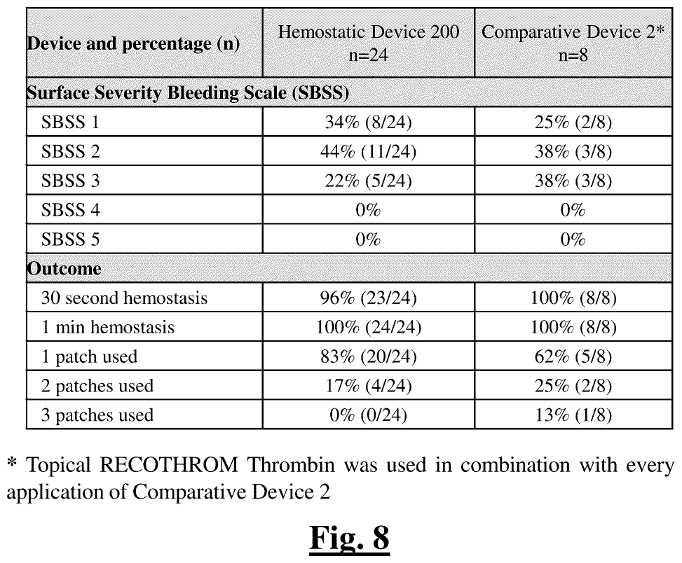

Four swine were enrolled and successfully underwent the implant procedure. Eight hepatic bleed sites with a minimal, mild or moderate bleeding were included per animal; 2 bleedings in one animal were excluded because a severe bleeding occurred as the result of the biopsy. Of the 32 included bleedings, 34% were minimal (SBSS 1), 44% were mild (SBSS 2), and 22% were moderate (SBSS 3) severity, respectively 38%, 46% and 17% for Hemostatic Patch 200 versus 25%, 38% and 38% for Comparative Device 2+Thrombin. The post-treatment SBSS evaluations at 30-40 seconds (targeting 30 seconds), and 1 minute±10 seconds, 3 minutes±10 seconds, and 5 minutes±10 seconds were based off the “final” appropriate treatment, not initial test article treatments of the site. Hemostatic Patch 200 reached a time-to-hemostasis of 30 seconds with the final patch placement in 96% of applications (n= 23/24) and 100% at 1 minute. One Hemostatic Patch 200 application required an additional 30 seconds of pressure and was hemostatic at 1 minute. Comparative Device 2+Thrombin reached hemostasis at 30 seconds in 100% of applications (n= 8/8). Four Hemostatic Patch 200 sites (n= 4/24, 17%) required a second Hemostatic Patch 200 application; the first Hemostatic Patch 200 adhered well to the tissue but did not fully cover the bleeding, and the second Hemostatic Patch 200 was applied partly overlapping the first Hemostatic Patch 200 and fully covering the bleeding. Three Comparative Device 2+Thrombin sites required a second or third Comparative Device 2+Thrombin application because applied patches failed to adhere to the tissue to reach hemostasis. A summary of the initial hemostasis and number of applications is provided in FIG. 8.

Of note, two bleeding sites with an SBSS of 4 were initially treated with coagulation, which was insufficient, and a (rescue) application of Hemostatic Patch 200—while the bleeding continued with an SBSS 4—resulted in hemostasis directly after a 30 second application time.

There was one rebleeding of a Hemostatic Patch 200 site 40 minutes after the initial application, caused by the site being damaged with a laparoscopic instrument while treating another lesion; an additional application of Hemostatic Patch 200 resolved the bleeding with a time to hemostasis of 30 seconds. No other rebleedings occurred. After a period of 30 minutes with normal intra-abdominal pressure, observation planned at the bleeding site and through the abdominal cavity found no rebleeding, no hematoma formation, and no product migration in any of the animals.

The usability of Hemostatic Patch 200 and Comparative Device 2+Thrombin was rated as acceptable for all introductions through a 15 mm trocar, acceptable for navigating to the bleeding site, acceptable for in-situ preparation for application on the bleeding site, and acceptable for application on the bleeding site.

At the time of the laparoscopic relook, all animals showed signs of overall gross adhesions. In 2 animals treated with Hemostatic Patch 200, approximately 25% of the liver adhered to the diaphragm; in one animal the left lateral lobe (LLL) adhered to the diaphragm, representing 4/8 treatment sites with adhesions, and in one animal the LLL adhered to the diaphragm, but there were no treatment sites (n= 0/8) on the LLL and thus there were 0/8 treatment sites with adhesions. In 1 animal treated with Hemostatic Patch 200 approximately 95% of the liver adhered to the diaphragm, representing to 8/8 sites with adhesions. In the 1 animal treated with Comparative Device 2+Thrombin, approximately 95% of the liver adhered to the diaphragm, representing 8/8 sites with adhesions. Therefore, adhesions at the treatment site were found in 50% after Hemostatic Patch 200 and 100% after Comparative Device 2+Thrombin. No animals showed signs of rebleeding, hematoma formation, or product migration.

At 4 weeks after treatment, gross adhesions were present in all Hemostatic Patch 200 and Comparative Device 2+Thrombin sites. An adhesion score of ‘1’ (thin, filmy adhesion; disrupted with minimal digital manipulation) was noted for 100% of Hemostatic Patch 200 and Comparative Device 2+Thrombin applications. None of the animals demonstrated any signs of rebleeding, hematoma formation or product migration. Visual signs of a device remnant were present in one of the Hemostatic Patch 200 sites (n= 1/24, 4%) versus 7 of the Comparative Device 2+Thrombin sites (n=⅞, 88%).

During follow-up, all animals recovered well from their surgeries. There was one significant finding in a pig treated with Hemostatic Patch 200, of a skin lesion on the ventral abdomen, at the caudal aspect of the incision, of an area ˜14×10 cm that is in a butterfly pattern. The incision itself was healed, and this area of skin is smooth, pink/purplish and has a darker purple-brown outline. It did not bother the pig, was non-painful, and non-pruritic. Blood work was unremarkable and the lesion improved without further treatment. The event was considered to be unrelated to the device.

### Comparison with GLP Open Implant Study

In both the open and laparoscopic GLP study, baseline bleeding severity was distributed across the range of minimal, mild, and moderate bleeding, and this distribution was similar, as shown in FIG. 8.

Four healthy porcine were successfully utilized on this study. A total of 24 Test article sites and 8 Control Article sites were treated. Two bleeding sites were created but not treated/assessed due to high baseline SBSS score (4). These sites were treated with a combination of cautery, which was insufficient to reduce the bleeding, and to stop the bleeding when it was still an SBSS of 4. Regarding pre-treatment SBSS scores for test article treatments, 37.5% (9 of 24) had a score of “1”, 45.8% (11 of 24) had a score of “2” and 16.7% (4 of 24) had a score of “3”. Regarding pre-treatment SBSS scores for control article treatments, 25.0% (2 of 8) had a score of “1”, 37.5% (3 of 8) had a score of “2” and 37.5% (3 of 8) had a score of “3”. All test and control article treatments were hemostatic, SBSS score of “0” at the 30-40 second post-treatment bleeding assessment and all test and control article treatments maintained hemostasis throughout the five-minute post-treatment assessment period, with the exception of test article treatment of lesion 3. For this animal, a SBSS score of “1” was observed at the 3 minute assessment, however the lesion was found to be hemostatic at the 5 minute assessment without any additional pressure time or additional product; it seems likely that this therefore represents a data collection error.

### Endpoint 1: Overall Animal Health (Moribundity)

Overall animal health (moribundity), defined as overall animal health, was assessed through review of physical examinations, clinical observations, clinical pathology, and medical treatments.

The success criteria established for Endpoint 1 was that there would be no clinically significant adverse event leading to early death or mortality due to treatment with the test articles. All assessments suggested that animals remained in good general health throughout the duration of the study. There were no deaths or major adverse events that affected animal health or welfare.

### Endpoint 2: Study Article Handling and Performance

Study article handling and performance was assessed by the Study Surgeon according to four parameters. The four parameters evaluated were: (1) Introduction through a trocar: acceptable if introduced in the body without sticking to the trocar, to the extent it can no longer be used on the bleeding site, (2) Navigation to bleeding site: acceptable if it doesn't stick to other organs/areas to the extent it can no longer be used on the bleeding site, while moving to the bleeding site, (3) Preparation for application: acceptable if patch can be unfolded and positioned at bleeding site and (4) Application: acceptable if placed on bleeding site and pressure applied with wet gauze. There were no success criteria established for Endpoint 2. However, the surgeon response to all criteria were positive, denoted as “acceptable” for both the test article (n=3) and control article (n=1) treated animals.

### Endpoint 3: Study Article Migration

Study article migration was assessed by visual migration and histopathology via light microscopy. Visual migration was assessed qualitatively by the assistant surgeon at the day 3 follow-up procedure and at the termination procedure. Histopathology, performed by the study pathologist, assessed sites on the liver and other tissue away from study article application for any indication of the study article migration.

No success criteria were established for the visual migration assessment, however, in summary there was no indication of test article (n=24) or control article (n=8) migration from any treatment sites at the day 3 and termination procedures. The success criteria regarding histopathology assessment of study article migration was no presence of test article found at non-treated sites collected for histology. Histopathology found no evidence of migrated study article in untreated liver sections.

### Endpoint 4: Hemostatic Success

Hemostatic success was assessed through evaluation of bleeding sites using the

Surface Bleeding Severity Scale (SBSS) prior to treatment, post-treatment, and at termination.

Also, hemostatic success was evaluated using visual assessment for re-bleeding at the end of treatment (prior to closure), at the day 3 follow-up procedure and at termination. The success criteria for endpoint 4 was that hemostasis (SBSS score of “0”) following application of the test article would be demonstrated to be effective without evidence of re-bleeding at termination. The SBSS scores prior to test and control article treatment ranged from 1-3. All test article treatments (n=24) and control article treatments (n=8) achieved hemostasis by the 5-minute post-treatment SBSS evaluation, with 16.7% of Hemostatic Patch 200 and 37.5% with control requiring additional product(s) to be placed for hemostasis, and there was no evidence of re-bleeding after the 30-minute return to normal intra-abdominal pressures on the day of treatment. Additionally, there was no evidence of re-bleeding at either the day 3 or termination procedures. Therefore, the success criteria for this endpoint was met.

### Endpoint 5: Time to Hemostasis

Time to hemostasis was defined as the time where a SBSS score of “0” was achieved and maintained through the post-treatment evaluation period. Bleeding was evaluated at each treatment site using the SBSS at 30 seconds, 1 minute, 3 minutes and 5 minutes after treatment with the test or control article. In instances where additional test/control article applications were required, the time to hemostasis was relative to the final test/control article application. For test article treatments, additional test article application was required in 4 of 24 treatments (16.7%) to achieve hemostasis (i.e., a second piece of patch for these cases), mainly because the patch was not fully covering the bleeding site. For control article treatments, additional control article application was required in 3 of 8 treatments (37.5%) (i.e., a second piece of patch in 2 cases and a third piece in 1 case), mainly because of no adhesion of the patch to the tissue. No success criteria were established for endpoint 5. However, in summary, for test article treatments, the time to hemostasis was 30 seconds for 95.8% (23 of 24) treatments and was 5 minutes for 4.2% (1 of 24) treatments. For control article treatments, the time to hemostasis was 30 seconds for 100% (8 of 8) treatments. Based on these results, the time to hemostasis was comparable between test and control article treatments.

### Endpoint 6: Adhesion Formation

Adhesion formation was evaluated and given an adhesion extent/severity score. Observed adhesions were also collected and evaluated for remnants of the study article and/or other tissue response. No success criteria were established for endpoint 6, however in summary, at termination all test and control article treatment sites received an adhesion score of “1”, which was defined as thin, filmy adhesion which could be disrupted with minimal digital manipulation. This suggests that adhesions were negligible to the test article treatment sites and the response was no different from the control article treatment sites.

### Endpoint 7: Local Tissue Response to the Study Articles

Local tissue response to the study articles was assessed at gross necropsy by evaluating study article treated tissues for any clinically significant abnormalities. At gross necropsy assessment 1 of 24 (4.2%) Test Article treatment sites were evaluated as having remaining study article present, while 7 of 8 (87.5%) Control Article treatment sites were evaluated as having remaining study article present. Additionally, local tissue response to the study articles was evaluated using histopathology evaluations of inflammatory response, mineralization, and amount of study article remaining at the treatment site. With histopathology, remaining Test Article was observed in 8/24 sections, where all 8 sites were observed to have 1-25% Test Article remaining compared to the amount at baseline. Examining this further, 1 site had ˜1%, 5 sites had ˜1-5%, 1 site had ˜5%, and 1 site had ˜5-10% of Test Article remaining. Mineralization was observed in 3/24 sections, foreign debris in 16/24 sections, and hemorrhage in 22/24 sections. Remaining Control Article was observed in ⅜ sections and was morphologically deeply basophilic, where 2 sites were observed to have 1-25% and 1 site to have 76-100% of Control Article compared to the amount at baseline. Mineralization was observed in ⅛ sections, foreign debris in 6/8 sections, and hemorrhage in ⅞ sections. No success criteria were established for endpoint 7, however, in summary, all recorded gross lesions likely reflect regions of inflammation and fibrosis and are expected findings given the model. Mononuclear cell infiltrates are also common background findings in pigs and are not considered to be related to the Test Article. Similar amounts of inflammation (and reactivity scores), mineralization, foreign debris, and hemorrhage were observed between Control and Test Article sites. There were no significant or unexpected findings within the hepatic lymph nodes. Histologic evidence of remaining Test Article was observed in similar rates between the Test Article (33.3%) and Control Article (37.5%), however the amounts of Test Article remaining were lower than the amounts of Control Article remaining.

In both the open and laparoscopic GLP study, Hemostatic Patch 200 and Comparative Device 2+Thrombin resulted in hemostasis at all bleeding sites: Hemostatic Patch 200 achieved hemostasis in 96% at 30 seconds and in 100% at 60 seconds in the laparoscopic GLP study versus 98.8% at 60 seconds and 100% at 2 minutes in the open GLP study. In the laparoscopic GLP study, there was the need for additional patches placed in both the Hemostatic Patch 200 (17%) and Comparative Device 2+Thrombin (38%) groups; rates for the requirement of additional patches for initial hemostasis in the open GLP study was n=0 for Hemostatic Patch 200 and n=2 for Comparative Device 2+Thrombin. In the Hemostatic Patch 200 group, this was because the initial Hemostatic Patch 200 did not cover the entire bleeding site; this was not observed in the open GLP study. In the Comparative Device 2+Thrombin group, this was because the patch failed to adhere well enough to the tissue; this was also observed in the open GLP study in one bleeding site (n= 1/80, 1.3%).

Despite hemostasis being achieved with both therapies and no rescue therapies were required, there was a larger need for additional patch placement in the laparoscopic GLP study versus the open GLP study for both the Hemostatic Patch 200 and Comparative Device 2+Thrombin groups. This is likely caused by the small size of the patches in relation to the biopsy punch size (e.g., a 2.5 cm by 2.5 cm patch on an 8 mm biopsy punch results in an overlap of non-bleeding tissue of only 8.5 mm if placed perfectly, unlike the recommended 1 cm in the instructions for use of Hemostatic Patch 200); although the size of the patch was similar in the open GLP study, the open surgical approach allows for better visualization and more direct placement of the patches. Moreover, application in the laparoscopic setting is generally believed to be more difficult because the view and angle on the bleeding site is not always optimal. Moreover, the surgeon performing the surgery does not have current routine experience with laparoscopic surgery and some degree of imprecise application can therefore be expected. Indeed, an additional minimally invasive studies on Hemostatic Patch 200 found that in the acute laparoscopic non-GLP study performed by a surgeon that routinely performs laparoscopic surgery, a same-sized 2.5 by 2.5 cm Hemostatic Patch 200 was placed appropriately overlapping the bleeding site in all 16 of 16 same-sized 8 mm biopsy punch bleedings. Of note, in an additional study on use of Hemostatic Patch 200 in a robotic partial liver resection model, where the surgeon chose to apply larger parts of Hemostatic Patch 200 (range of 3 by 5 cm to 10 by 5 cm) with a sufficient overlap on clinically representable bleedings, all applications appropriately covered the bleeding site.

The completion of a usability questionnaire by 7 surgeons that performed different minimally invasive procedures with GATT-Patch in the present studies found that all surgeons answered “Strongly agree” or “Agree” on a 5-Point Likert scale on the following statements about Hemostatic Patch 200:
Hemostatic Patch 200 could be introduced through a trocar without damaging, fragmenting, or rolling up to the degree that it can no longer be used (n=6 “Strongly Agree” and n=1 “Agree”)
Hemostatic Patch 200 could be navigated to the bleeding site without sticking to other structures or becoming too wet, to the degree that it can no longer be used (n=6 “Strongly Agree” and n=1 “Agree”)
Hemostatic Patch 200 could be adequately positioned on the bleeding site with minimally invasive tools (n=7 “Strongly Agree”)
Sufficient pressure could be provided with minimally invasive tools to adhere Hemostatic Patch 200 to the tissue (n=7 “Strongly Agree”).

In the open GLP study, there was only one case of rebleeding after Hemostatic Patch 200 application (e.g. 1.3%) that occurred during the procedure. In the laparoscopic GLP study there were no rebleedings occurring because of a hemostatic failure of Hemostatic Patch 200. There was one unintentional injury to a treatment site with a laparoscopic instrument that required a new Hemostatic Patch 200 application 40 minutes after the initial treatment. Of note, the assessment of rebleeding in the laparoscopic GLP Study included a 30-minute period of normal intra-abdominal pressure to mimic the direct postoperative period during which the risk of rebleeding is highest, and no rebleeding occurred during this period. There was no evidence of rebleeding at the 72-hrs sacrifice in the open GLP study and the 72-hrs laparoscopic relook in the laparoscopic GLP study, nor was there evidence of rebleeding at later timepoints of 4 and 8 weeks in both studies. In summary, the laparoscopic GLP study confirmed that in a minimally invasive setting the use of Hemostatic Patch 200 resulted in persistent hemostasis in a similar degree as when Hemostatic Patch 200 was used during open surgery.

In both the open and laparoscopic GLP studies, there was no evidence of product migration at any of the timepoints during surgery, at the 72-hrs sacrifice and laparoscopic relook, and at the later 4-and 8-week timepoints. The data from the laparoscopic GLP study provides evidence on appropriate pressure being applied during the application of Hemostatic Patch 200 so that it adequately adheres to the tissue and does not migrate, similar to the results in the open GLP study.

In the open GLP study, 72-hrs sacrifice identified overall gross adhesions to be present in 81% of Hemostatic Patch 200 sites and 86% of Comparative Device 2+Thrombin sites, as compared with 50% of Hemostatic Patch 200 sites and 100% of Comparative Device 2+Thrombin sites in the laparoscopic GLP study. In the open GLP study, these adhesions were all graded as ‘1’ (thin, filmy adhesion; disrupted with minimal digital manipulation). Because of the laparoscopic approach of the 3-day relook in the laparoscopic GLP study, no scoring of the nature of the adhesions was performed at this time. At the 4-week endpoint, the open GLP study found that gross adhesions were present in all Hemostatic Patch 200 and Comparative Device 2+Thrombin sites, and this was also found in the laparoscopic GLP study. The nature of the adhesions suggests that the laparoscopic GLP study was associated with a lower adhesions grading at the 4-week endpoint than the open GLP study. Of note, the open GLP study found at 8 weeks sacrifice that 83.3% of Hemostatic Patch 200 and 91% of Comparative Device 2+Thrombin sites had adhesion scores of ‘2’ and 8.3% and 9.0%, respectively, with a score of ‘3’, demonstrating that long-term adhesions are not expected to be more with Hemostatic Patch 200 versus Comparative Device 2+Thrombin.

With the majority of animals showing adhesions in the open and laparoscopic GLP study, irrespective of which hemostatic treatment is used, and overall clinical evidence on the existence of adhesions after intra-abdominal surgery for different indications, it can be summarized that Hemostatic Patch 200 by itself does not appear to be associated with an increase of adhesions post-surgery. Visual (gross) inspection of treatment sites at 4 weeks in the open and laparoscopic GLP study showed that the majority of treatment sites did not show any remnants of Hemostatic Patch 200, with rates of possible remnants being visible of 21% in the open GLP study and 4% in the laparoscopic GLP study.

During histopathology, it was confirmed in the open GLP study that degradation of Hemostatic Patch 200 occurred before the 4-week sacrifice of animals as there were no microscopical signs of Hemostatic Patch 200 on the bleeding sites.

The results from the laparoscopic GLP study confirm that Hemostatic Patch 200 can be satisfactorily introduced through a minimally invasive trocar, navigated to the bleeding site, and applied to the bleeding; the surgeon rates all data elements regarding usability as ‘acceptable’. As compared to Hemostatic Patch 200 use in an open surgical setting, laparoscopic use of Hemostatic Patch 200 demonstrated similar performance to the open GLP study in terms of hemostasis, rebleeding, product migration, adhesion formation, and product degradation. Based on these initial results, minimally invasive use of Hemostatic Patch 200 is considered safe and effective.

### Study 4: Non-GLP Study with Hemostatic Patch 200 in Robotic Liver Resection

The surgery was performed by a consulting surgeon that routinely performs robotic liver surgery. Two pigs were used during this terminal experiment. Lesions of the liver parenchyma and the main vessels of the liver were created by using sharp non-cauterizing scissors, sharp bipolar-cauterizing scissors, or bipolar vessel sealing system. A series of 6 lesions were aimed per animal. Lesions made on the main blood vessels are not mentioned in this summary as those are out-of-scope of the current regulatory submission.

Per protocol, the majority of lesions were treated with Hemostatic Patch 200. Before introduction in the trocar, Hemostatic Patch 200 was cut to the appropriate size so that it overlapped non-bleeding tissue by at least 1 cm on all sides, according to the IFU. Hemostatic Patch 200 was subsequently rolled over its long axis and grasped at the most distal tip to be introduced through a 12 mm size trocar. After navigation to the bleeding and unrolling, Hemostatic Patch 200 was positioned on the bleeding and applied by 30 seconds of pressure with a saline-wetted gauze, per IFU, after which hemostasis was checked by careful removal of the gauze. If hemostasis was not yet achieved, either additional pressure, or additional (pieces of) Hemostatic Patch 200 were applied. Hemostasis was checked at predetermined times of 30 seconds, 1 minute, and 3 minutes. The time to hemostasis was considered to be the time that hemostasis was achieved and maintained. For reasons of a comparative feasibility, one lesion was treated with Comparative Device 1. Comparative Device 1 was cut to the appropriate size so that it overlapped non-bleeding tissue by at least 1 cm on all sides. It was subsequently folded and placed inside a cut-off finger from a surgical glove after which it was grasped at the most distal tip and introduced through a 12 mm trocar. After unpacking out of the glove and unfolding, it was navigated to the bleeding. After positioning on the bleeding, it was applied per IFU by 3 minutes of pressure with a saline wetted gauze. Hemostasis was checked at 3 minutes. If hemostasis was not yet achieved, either additional pressure, or additional (pieces of) Comparative Device 1 were applied.

The primary outcome was hemostatic performance as determined by the time-to hemostasis. Furthermore, the usability of Hemostatic Patch 200, in terms of preparation for introduction through a trocar, the introduction through a trocar, the navigation to the bleeding site, the unrolling and positioning on the bleeding site, the application of the patch with a wet gauze, and the removal of the gauze after application, were all scored as “pass” if successful without rendering the patch to be unusable, or “fail” if the patch could no longer be used.

A total of 10 lesions were created which were initially treated with Hemostatic Patch 200 (n=9) and Comparative Device 1 (n=1). In the second animal, the blood pressure could not be maintained above a MAP of 60 mm Hg because of severe blood loss during the procedure already-partly due to the lesion treated with Comparative Device 1 requiring rescue treatment with Hemostatic Patch 200—the procedure was concluded after treatment of the 4th lesion. Two lesions are excluded from the current summary as these were lesions of the main vessels and are out-of-scope of the current regulatory submission.

Of the 8 parenchymatous lesions, hemostasis with Hemostatic Patch 200 (n=8) was achieved with a 30-seconds application time in 88% (n=⅞). One lesion required an additional Hemostatic Patch 200 application and resulted in hemostasis at the 8-minute time point. One lesion was initially treated with Comparative Device 1 and hemostasis was not achieved with 2 applications of Comparative Device 1; Comparative Device 1 was removed and the lesion was subsequently treated with Hemostatic Patch 200 and resulted in hemostasis with a 30 seconds application time (this lesion is included as one of the 8 mentioned above).

On the right medial lobe of the liver, on which no blood inflow reduction was performed, a resection over the length of 6 cm and a depth and height of, respectively, 2 cm and 3 cm was performed using the sharp bipolar cauterizing scissors. The bleeding severity was an SBSS of 3 (e.g. moderate). A full 10×5 cm Hemostatic Patch 200 was rolled and introduced through the trocar and positioned on the bleeding. After a 30 seconds application time with a wet gauze, the gauze was removed without adhesion to the patch and hemostasis was achieved and persisted through all timepoints.

On the left lateral lobe of the liver, on which no blood inflow reduction was performed, a deep metastasectomy was performed with a depth of 2 cm and a diameter of 2.5 cm, using the sharp non-cauterizing scissors (after marking the resection with cauterization). The bleeding severity was an SBSS of 4 (e.g., severe). A full 10×5 cm Hemostatic Patch 200 was rolled and introduced through the trocar and positioned in the depth of the bleeding, with an additional half a Hemostatic Patch 200 (e.g., 5×5 cm) placed on top of the bleeding. After a 30 seconds application time with a wet gauze, the gauze was removed without any adhesion of the gauze to the patch. Hemostasis was achieved and persisted through all timepoints.

A resection of 3 cm and a depth and height of, respectively, 3 cm and 2 cm was performed using sharp non-cauterizing scissors. The bleeding severity was SBSS 5 (e.g. extreme). About a 7×5 cm piece of Hemostatic Patch 200 was positioned on the bleeding as a plug of patch without unrolling. This was done because of the severity of the bleeding the surgeon did not further optimize the camera position and opted for applying pressure with a piece of patch as soon as possible without assuring adequate positioning of the patch, to already reduce the initial SBSS 5 bleeding to a lower grade. Furthermore, because of the severity of bleeding, the wet gauzes were already drenched with blood when applying on the patch. Lastly, the suction tube was clogged with tissue and no adequate drainage of the bleeding could be performed. These factors led to the patch being on the tissue for an initial 3 min and 30 seconds. Likely because of these reasons, when the gauzes were removed, a piece of the patch was removed with the gauze. The bleeding persisted with an SBSS of 2 and required a second 5×5 cm sized piece of Hemostatic Patch 200 to be placed partly overlapping the previous patch (after unrolling in this case) and covering the bleeding site. After 30 seconds application over the entire surface of the patch, the gauzes could easily be removed and hemostasis was achieved and persisted. The overall time-to-hemostasis, including the initial application+the removal of the gauze+better positioning of the camera position+additional suction to achieve good visibility of the bleeding site+preparation and introduction and positioning of the additional piece of Hemostatic Patch 200, was 8 minutes.

For comparison with the standard of care treatment in this type of surgery, it was decided to perform a resection and treat this bleeding with Comparative Device 1. Because Comparative Device 1 requires preparation before it can be used minimally invasively and creating a bleeding before preparation would cause unnecessary blood loss in this preclinical test, a full size Comparative Device 1 of 10×5 cm was folded and placed in a cut-off finger of a surgical glove, and subsequently introduced through the trocar. During introduction, a small part of Comparative Device 1 that was outside of the surgical glove broke off and remained in the trocar; the size was now 8×5 cm. After introduction of Comparative Device 1, a resection was created with the sharp non-cauterizing scissors on the right medial lobe. The size of the resection was a length of 3 cm and a depth and height of, respectively, 3 cm and 2 cm. The bleeding severity was an SBSS of 4 (e.g. severe). The Comparative Device 1 was removed from the glove with further breaking off of several active yellow components of the patch, and subsequently positioned onto the bleeding. Pressure with a wet gauze was initiated for 3 minutes.

After removal of the gauze, partly removing the Comparative Device 1 from the bleeding site with it, it was clear that there was no hemostasis. A second 5×5 cm Comparative Device 1 was introduced in the same method as the first patch and placed onto the area of the bleeding site that continued to bleed. Again, 3 minutes of pressure was performed. After removal of the gauze, hemostasis seemed to have occurred, but within the next several minutes, breakthrough bleeding at the edges of the Comparative Device 1 increasingly occurred. Eventually, the entire patch was removed from the bleeding and a full-size Hemostatic Patch 200 was placed on the bleeding. After 30 seconds of pressure with a wet gauze, the gauze was removed without adhesion to the patch and hemostasis was achieved and persisted through the remainder of the timepoints.

### Minimally Invasive Usability

In nearly all cases requiring different sizes of Hemostatic Patch 200 ranging from about 3×5 cm to a full Hemostatic Patch 200 of 10×5 cm, the patch could be rolled over its long axis easily and introduced through a 12 mm trocar without damage to the patch; only once was there a tear in a small piece of Hemostatic Patch 200 that was quickly folded for introduction instead of rolled. After introduced, Hemostatic Patch 200 could be navigated to the bleeding site without rendering it unusable because of adhering to other structures or to the surgical instruments. It could be unrolled and positioned on the bleeding site and subsequent pressure with a wet gauze caused the patch to be sufficiently adherent to the tissue in the majority of cases, thereby causing hemostasis to occur quickly after 30 seconds of pressure and maintaining its hemostatic performance through further timepoints. The Hemostatic Patch 200 specific usability questionnaire completed by the surgeon after the procedure showed that the surgeon “strongly agreed” with statements about user-friendliness.

Hemostasis of up to life-threatening bleeding during robotic liver resection can be achieved quickly and reliably with Hemostatic Patch 200. In addition to the hemostatic performance of Hemostatic Patch 200, the lack of preparation and ease-of-use even in minimally invasive settings may help reduce the need for conversion to open surgery to control bleedings, and thereby significantly benefit patient outcomes. This data provides evidence on several important aspects of Hemostatic Patch 200:

1. Hemostatic Patch 200, in its full size of 10×5 cm, when rolled over its long axis, could be easily introduced without damaging the patch, through a trocar size of 12 mm. Furthermore, in all cases, Hemostatic Patch 200 was easily navigated to the bleeding and positioned onto the bleeding with adequate pressure upon application. This is also reflected in the usability questionnaire completed by the independent surgeon that performed the procedure;

2. Hemostatic Patch 200 could be used during a variety of bleeding scenarios, representing also challenging bleedings requiring a flexible and pliable product for adequate positioning of the bleeding site;

3. Even in challenging minimally invasive settings, the 30-seconds application time of Hemostatic Patch 200, as described in its IFU, is adequate in achieving hemostasis in the vast majority of cases of soft tissue (e.g. in this case liver) bleeding (e.g. 88%), similar to what is achieved in other open and minimally invasive preclinical and (open) clinical studies, as further elaborated upon in this document;

4. While the initial indication for Hemostatic Patch 200 is proposed to be for use on minimal, mild, and moderate bleeding, this study supports use of Hemostatic Patch 200 in severe bleedings for potential future regulatory approvals;

5. Although only tested once against the standard-of-care hemostatic patch, Hemostatic Patch 200 appears to have ease-of-use and performance advantages over Comparative Device 1 in the minimally invasive setting.

The hemostatic device 200 and related methods of use of this disclosure demonstrated high rates of substantial hemostasis in patients with hemorrhage during minimally invasive procedures. The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

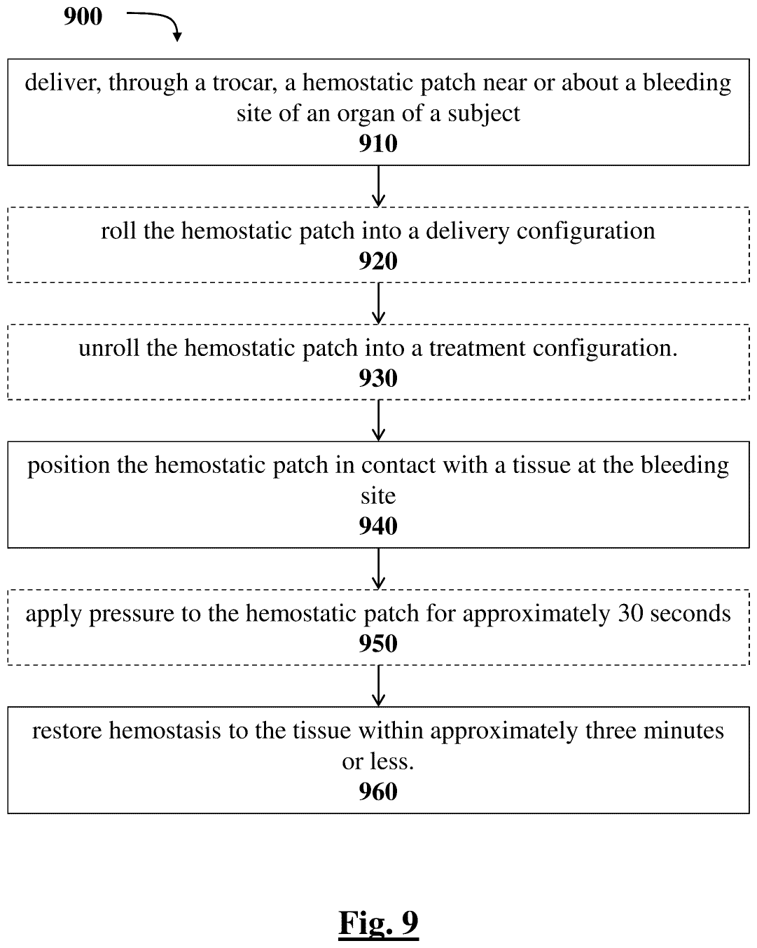

FIG. 9 depicts a method 900 for treating hemorrhage in a subject during a minimally invasive procedure. The method 900 can include delivering, through a trocar, a Hemostatic Patch 200 near or about a bleeding site of an organ of a subject at step 910. Optionally, Hemostatic Patch 200 can be rolled into a delivery configuration at step 920. Rolling Hemostatic Patch 200 along the long edge may produce a delivery configuration that allows for delivery through a trocar having a diameter of ranging from about 10 mm to about 36 mm. Method 900 can further optionally include unrolling the Hemostatic Patch 200 into a treatment configures at step 930. After delivery through the trocar, method 900 includes positioning Hemostatic Patch 200 in contact with a tissue at the bleeding site at step 940. Although not depicted in method 900, the operator can further position Hemostatic Patch 200 within a cavity at the bleeding site. Method 900 can optionally include applying pressure to Hemostatic Patch 200 for approximately 30 seconds at step 950. Method 900 further includes restoring hemostasis to the tissue within approximately three minutes or less at step 960.

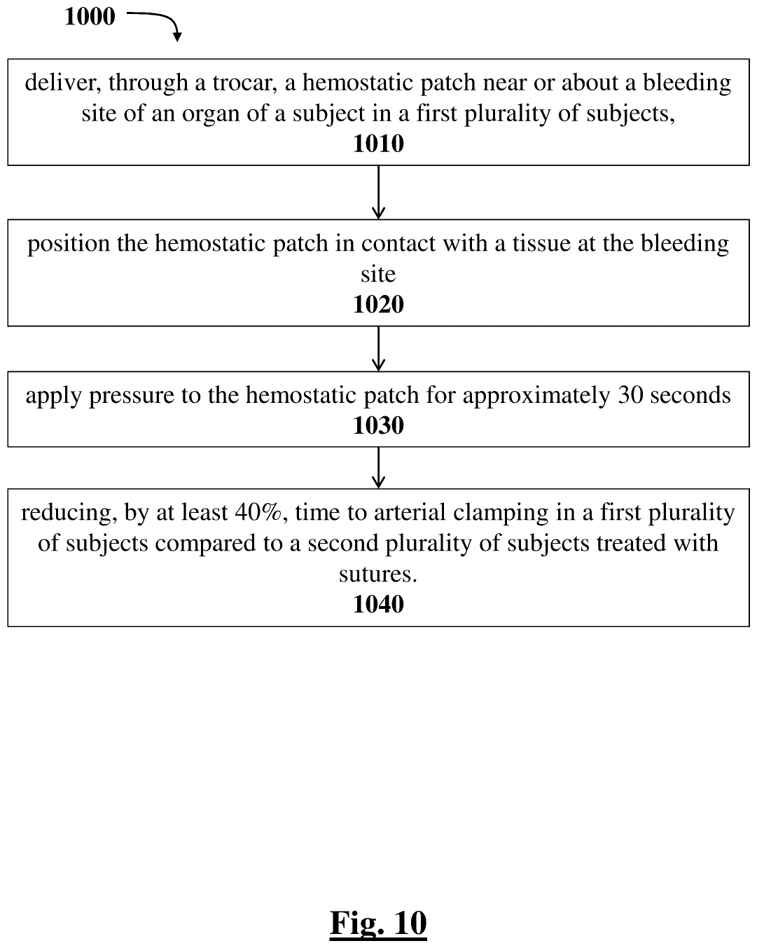

FIG. 10 depicts a method 1000 for treating hemorrhage in a subject during a minimally invasive procedure. The method 1000 can include delivering, through a trocar, a Hemostatic Patch 200 near or about a bleeding site of an organ of a subject at step 1010. After delivery through the trocar, method 1000 includes positioning Hemostatic Patch 200 in contact with a tissue at the bleeding site at step 1020. Although not depicted in method 1000, the operator can further position Hemostatic Patch 200 within a cavity at the bleeding site. Method 1000 includes applying pressure to Hemostatic Patch 200 for approximately 30 seconds at step 1030. Method 1000 further includes reducing, by at least 40%, time of arterial clamping in a first plurality of subjects compared to a second plurality of subjects treated with sutures at step 1040. As described supra method 1000 can also include rolling Hemostatic Patch 200 into a delivery configuration and unrolling Hemostatic Patch 200 into a treatment configuration between steps 1010 and 1020.

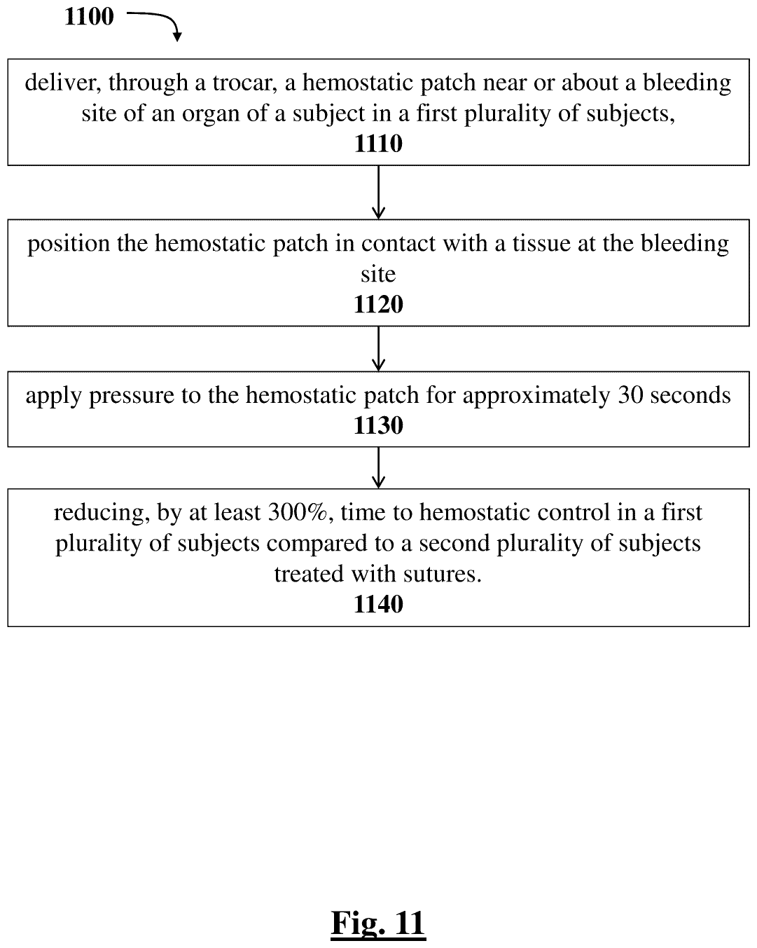

FIG. 11 depicts a method 1100 for treating hemorrhage in a subject during a minimally invasive procedure. The method 1100 can include delivering, through a trocar, a Hemostatic Patch 200 near or about a bleeding site of an organ of a subject at step 1110. After delivery through the trocar, method 1100 includes positioning Hemostatic Patch 200 in contact with a tissue at the bleeding site at step 1120. Although not depicted in method 1100, the operator can further position Hemostatic Patch 200 within a cavity at the bleeding site. Method 1100 includes applying pressure to Hemostatic Patch 200 for approximately 30 seconds at step 1130. Method 1100 further includes reducing, by at least 300%, time to hemostatic control in a first plurality of subjects compared to a second plurality of subjects treated with sutures at step 1140. As described supra method 1100 can also include rolling Hemostatic Patch 200 into a delivery configuration and unrolling Hemostatic Patch 200 into a treatment configuration between steps 1110 and 1120.

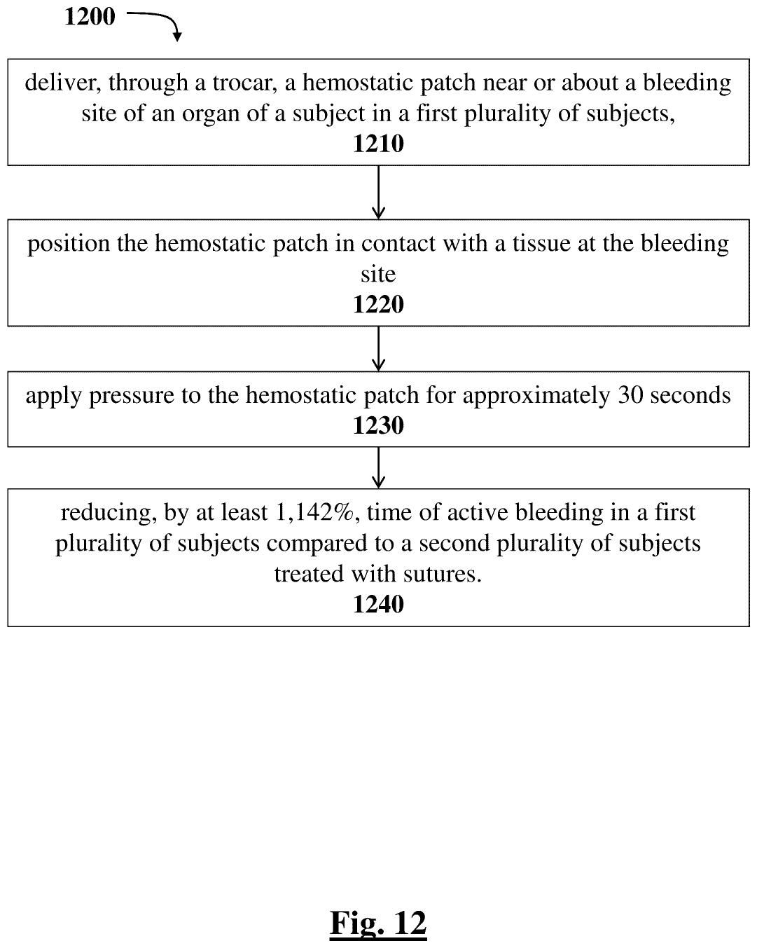

FIG. 12 depicts a method 1200 for treating hemorrhage in a subject during a minimally invasive procedure. The method 1200 can include delivering, through a trocar, a Hemostatic Patch 200 near or about a bleeding site of an organ of a subject at step 1210. After delivery through the trocar, method 1200 includes positioning Hemostatic Patch 200 in contact with a tissue at the bleeding site at step 1220. Although not depicted in method 1200, the operator can further position Hemostatic Patch 200 within a cavity at the bleeding site. Method 1200 includes applying pressure to Hemostatic Patch 200 for approximately 30 seconds at step 1230. Method 1200 further includes reducing, by at least 1.142%, time of active bleeding in a first plurality of subjects compared to a second plurality of subjects treated with sutures at step 1240. As described supra method 1200 can also include rolling Hemostatic Patch 200 into a delivery configuration and unrolling Hemostatic Patch 200 into a treatment configuration between steps 1210 and 1220.

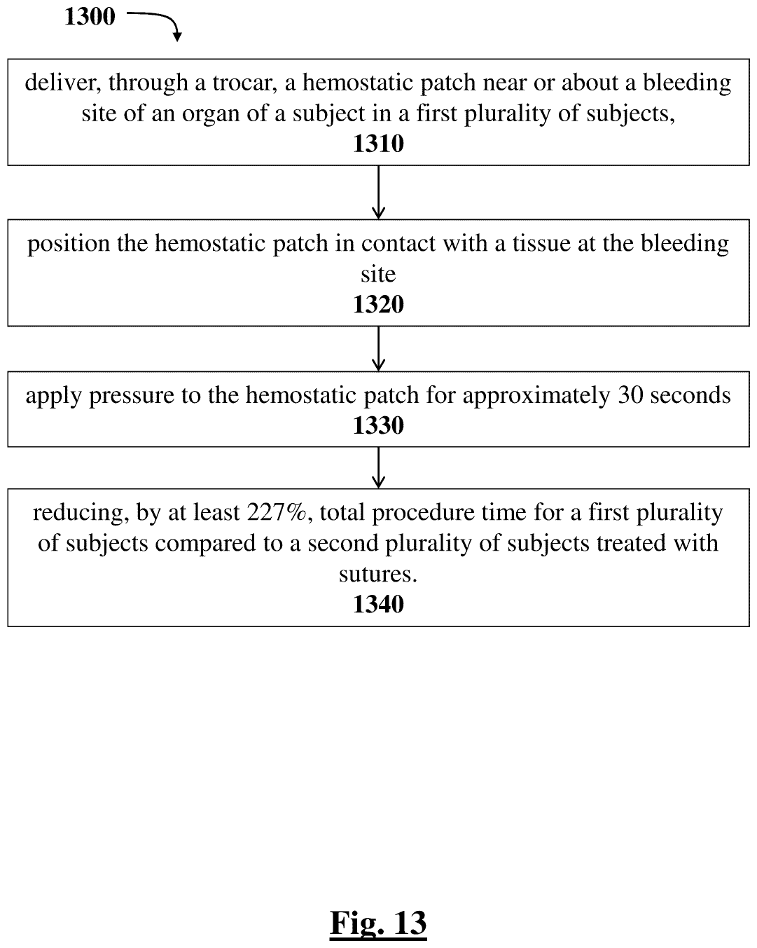

FIG. 13 depicts a method 1300 for treating hemorrhage in a subject during a minimally invasive procedure. The method 1300 can include delivering, through a trocar, a Hemostatic Patch 200 near or about a bleeding site of an organ of a subject at step 1310. After delivery through the trocar, method 1300 includes positioning Hemostatic Patch 200 in contact with a tissue at the bleeding site at step 1320. Although not depicted in method 1300, the operator can further position Hemostatic Patch 200 within a cavity at the bleeding site. Method 1300 includes applying pressure to Hemostatic Patch 200 for approximately 30 seconds at step 1330. Method 1300 further includes reducing, by at least 227%, total procedure time for a first plurality of subjects compared to a second plurality of subjects treated with sutures at step 1340. As described supra method 1300 can also include rolling Hemostatic Patch 200 into a delivery configuration and unrolling Hemostatic Patch 200 into a treatment configuration between steps 1310 and 1320.

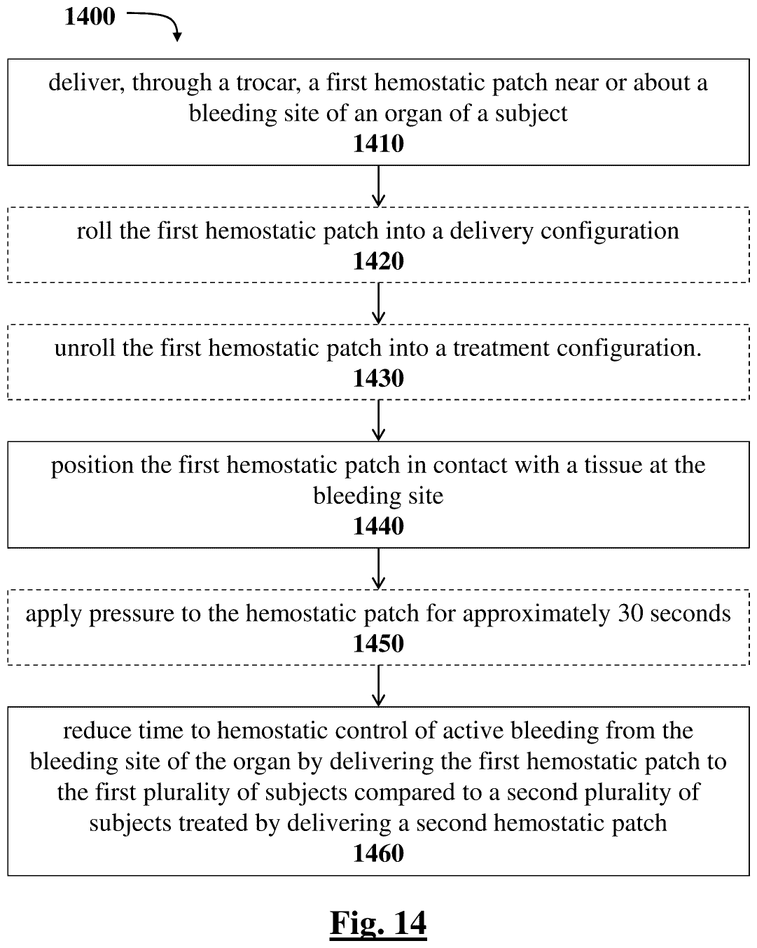

FIG. 14 depicts a method 1400 for treating hemorrhage in a subject during a minimally invasive procedure. The method 1400 can include delivering, through a trocar, a Hemostatic Patch 200 near or about a bleeding site of an organ of a subject at step 1410. Optionally, Hemostatic Patch 200 can be rolled into a delivery configuration at step 1420. Rolling Hemostatic Patch 200 along the long edge may produce a delivery configuration that allows for delivery through a trocar having a diameter of ranging from about 10 mm to about 36 mm. Method 1400 can further optionally include unrolling the Hemostatic Patch 200 into a treatment configures at step 1430. After delivery through the trocar, method 1400 includes positioning Hemostatic Patch 200 in contact with a tissue at the bleeding site at step 1440. Although not depicted in method 1400, the operator can further position Hemostatic Patch 200 within a cavity at the bleeding site. Method 1400 can optionally include applying pressure to Hemostatic Patch 200 for approximately 30 seconds at step 1450. Method 1400 further includes reducing time to hemostatic control of active bleeding from the bleeding site of the organ by delivering the first hemostatic patch to the first plurality of subjects compared to a second plurality of subjects treated by delivering a second hemostatic patch at step 1460.

The disclosed technology described herein can be further understood according to the following clauses:

Clause 1: A method for treating hemorrhage in a subject during a minimally invasive procedure, the method comprising: delivering, through a trocar, a hemostatic patch near or about a bleeding site of an organ of a subject, the hemostatic patch comprising: a fibrous carrier structure, and reactive electrophilic groups capable of reacting with amine groups in tissue and blood; positioning the hemostatic patch in contact with a tissue at the bleeding site;

and restoring hemostasis to the tissue within three minutes or less.

Clause 2: The method of Clause 1, the hemostatic patch further comprising: a three-dimensional interconnected interstitial space comprising a plurality of reactive polymer particles comprising: an electrophilic polymer carrying the reactive electrophilic groups, and a nucleophilic cross-linking agent that contains reactive nucleophilic groups that are capable of reacting with the reactive electrophilic groups of the electrophilic polymer under the formation of a covalent bond.

Clause 3: The method of Clauses 1 or 2, further comprising rolling the hemostatic patch into a delivery configuration.

Clause 4: The method of Clause 1, the trocar comprising a diameter ranging from about 8 mm to about 16 mm.

Clause 5: The method of any of Clauses 1-4, further comprising unrolling the hemostatic patch into a treatment configuration.

Clause 6: The method of any of Clauses 1-5, further comprising applying pressure to the hemostatic patch positioned in contact with the tissue at the bleeding site, the pressure being applied for about 30 seconds.

Clause 7: The method of any of Clauses 1-6, further comprising: achieving, within approximately 30 seconds, hemostasis to the organ to a subject presenting a bleeding severity equal to or less than 2 at the bleeding site of the organ determined by a surface bleeding severity scale (SBSS).

Clause 8: The method of any of Clauses 1-6, further comprising: achieving, within approximately 93 seconds, hemostasis to the organ to a subject presenting a bleeding severity equal to or less than 4 at the bleeding site of the organ determined by a surface bleeding severity scale (SBSS).

Clause 9: The method of any of Clauses 1-6, further comprising: achieving, within approximately 72 seconds, hemostasis to the organ to a subject presenting a bleeding severity equal to or less than 5 at the bleeding site of the organ determined by a surface bleeding severity scale (SBSS).

Clause 10: The method of any of Clauses 1-9, wherein the bleeding site is located in one of the following locations: liver, pancreas, spleen, stomach, gastrointestinal tract, kidney, bladder, reproductive organs, lungs, mediastinum, breast, lymph nodes, thymus, muscles, fat, heart, blood vessels, iliac arteries, carotid artery, vena cava, or brain.

Clause 11: The method of any of Clauses 1-10, the hemostatic patch configured to fully degrade within approximately six weeks.

Clause 12: The method of any of Clauses 1-11, further comprising allowing degradation of the hemostatic patch after restoring hemostasis to the organ.

Clause 13: The method of any of Clauses 1-12, further comprising providing the reactive electrophilic groups from the electrophilic polymer and the reactive nucleophilic groups from a nucleophilic polymer.

Clause 14: The method of Clause 13, wherein the electrophilic polymer is selected from polyoxazolines, polyethylene glycols, polyvinylpyrrolidones, polyurethanes and combinations thereof.

Clause 15: The method of Clause 14, wherein the electrophilic polymer is a polyoxazoline.

Clause 16: The method of any of Clauses 1-15, wherein the reactive electrophilic groups are selected from the group consisting of carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, olefins, glycidyl ethers, carboxyl, succinimidyl esters, sulfo succinimidyl esters, maleimido (maleimidyl), ethenesulfonyl, imido esters, aceto acetate, halo acetal, orthopyridyl disulfide, dihydroxy-phenyl derivatives, vinyl, acrylate, acrylamide, iodoacetamide and combinations thereof.

Clause 17: The method of Clause 13, the hemostatic patch comprising a molar ratio of electrophilic polymer to the nucleophilic polymer ranging from about 1.0:0.10 to about 1.0:0.40.

Clause 18: A method for treating hemorrhage in a subject during a minimally invasive procedure, the method comprising: delivering, through a trocar, a hemostatic patch near or about a bleeding site of an organ of the subject, the hemostatic patch comprising: a nucleophilic polymer carrying reactive nucleophilic groups, and an electrophilic polymer carrying at least three reactive electrophilic groups capable of reacting with the nucleophilic polymer and amine groups in tissue and blood; positioning the hemostatic patch in contact with a tissue at the bleeding site of the organ; applying pressure to the hemostatic patch for approximately 30 seconds; and restoring hemostasis of the organ within approximately three minutes or less.

Clause 19: The method of Clause 18, further comprising: reducing, by at least 40%, time of arterial clamping in a first plurality of subjects after positioning the hemostatic patch in contact with the tissue at the bleeding site of the organ and applying pressure for approximately 30 seconds in a respective subject, compared to a second plurality of subjects treated with sutures, the first plurality of subjects presenting an average bleeding severity equal to or less than 5 at the bleeding site of the organ determined by a surface bleeding severity scale (SBSS).

Clause 20: The method of Clause 18, further comprising: reducing, by at least 300%, time to hemostatic control in a first plurality of subjects after positioning the hemostatic patch in contact with the tissue at the bleeding site of the organ and applying pressure for approximately 30 seconds in a respective subject, compared to a second plurality of subjects treated with sutures, the first plurality of subjects presenting an average bleeding severity equal to or less than 3 at the bleeding site of the organ determined by a surface bleeding severity scale (SBSS).

Clause 21: The method of Clause 18, further comprising: reducing, by at least 1.142%, time of active bleeding in a first plurality of subjects after positioning the hemostatic patch in contact with the tissue at the bleeding site of the organ and applying pressure for approximately 30 seconds in a respective subject, compared to a second plurality of subjects treated with sutures, the first plurality of subjects presenting an average bleeding severity equal to or less than 3 at the bleeding site of the organ determined by a surface bleeding severity scale (SBSS).

Clause 22: The method of Clause 18, further comprising: reducing, by at least 227%, total procedure time for a first plurality of subjects after positioning the hemostatic patch in contact with the tissue at the bleeding site of the organ and applying pressure for approximately 30 seconds in a respective subject, compared to a second plurality of subjects treated with sutures, the first plurality of subjects presenting an average bleeding severity equal to or less than 3 at the bleeding site of the organ determined by a surface bleeding severity scale (SBSS).

Clause 23: The method of any of Clauses 18-22, wherein the bleeding site is located in one of the following locations: liver, pancreas, spleen, stomach, gastrointestinal tract, kidney, bladder, reproductive organs, lungs, mediastinum, breast, lymph nodes, thymus, muscles, fat, heart, blood vessels, iliac arteries, carotid artery, vena cava, or brain.

Clause 24: The method of Clause 18, further comprising: providing a sheet of water-resistant cohesive fibrous carrier structure comprising a three-dimensional interconnected interstitial space and fibers comprising the nucleophilic polymer carrying reactive nucleophilic groups; providing reactive polymer particles comprising the electrophilic polymer comprising at least three reactive electrophilic groups that are capable of reacting with both amine groups in tissue and blood and with the reactive nucleophilic groups of the nucleophilic polymer to form covalent bonds; and distributing the reactive polymer particles within the interstitial space of the fibrous carrier structure in an amount of at least 3% by weight of the fibrous carrier structure for form the hemostatic patch.

Clause 25: The method of any of Clauses 18-24, further comprising: reducing, by delivering the hemostatic patch to a first plurality of subjects, time to hemostatic control of active bleeding from the bleeding site of the organ compared to a second plurality of subjects treated by suturing the bleeding site of the organ.

Clause 26: The method of Clause 25, further comprising: reducing time to hemostatic control by at least 1 minute and 7 seconds, by delivering the hemostatic patch to the first plurality of subjects.

Clause 27: The method of Clause 25, further comprising: reducing time to hemostatic control by an average of approximately 6 minutes and 28 seconds, by delivering the hemostatic patch to the first plurality of subjects.

Clause 28: The method of Clause 18, further comprising: reducing, by delivering the hemostatic patch to a first plurality of subjects, time of active bleeding from the bleeding site of the organ during hemostatic control compared to a second plurality of subjects treated by suturing the bleeding site of the organ.

Clause 29: The method of Clause 28, further comprising: reducing time of active bleeding from the bleeding site of the organ during hemostatic control by at least 4 seconds, by delivering the hemostatic patch to the first plurality of subjects.

Clause 30: The method of Clause 28, further comprising: reducing time of active bleeding from the bleeding site of the organ during hemostatic control by an average of approximately 5 minutes and 23 seconds, by delivering the hemostatic patch to the first plurality of subjects.

Clause 31: The method of Clause 18, further comprising: reducing, by delivering the hemostatic patch to a first plurality of subjects, total procedure time of treating hemorrhage in the first plurality of subjects compared to a second plurality of subjects treated by suturing the bleeding site of the organ.

Clause 32: The method of Clause 31, further comprising: reducing total procedure time by at least 1 minute and 11 seconds, by delivering the hemostatic patch to the first plurality of subjects.

Clause 33: The method of Clause 31, further comprising: reducing total procedure time by an average of approximately 6 minutes and 59 seconds, by delivering the hemostatic patch to the first plurality of subjects.

Clause 34: A method for treating hemorrhage in a subject during a minimally invasive procedure, the method comprising: delivering, through a trocar, a first hemostatic patch near or about a bleeding site of an organ of a respective subject in a first plurality of subjects, the first hemostatic patch comprising: a nucleophilic polymer carrying reactive nucleophilic groups, and an electrophilic polymer carrying at least three reactive electrophilic groups capable of reacting with the nucleophilic polymer and amine groups in tissue and blood; positioning the first hemostatic patch in contact with a tissue at the bleeding site; applying pressure to the first hemostatic patch for approximately 30 seconds; and reducing time to hemostatic control of active bleeding from the bleeding site of the organ by delivering the first hemostatic patch to the first plurality of subjects compared to a second plurality of subjects treated by delivering a second hemostatic patch.

Clause 35: The method of Clause 34, further comprising rolling the first hemostatic patch into a delivery configuration.

Clause 36: The method of Clauses 34 or 35, further comprising unrolling the first hemostatic patch into a treatment configuration.

Clause 37: The method of Clause 34, further comprising: restoring, by the first hemostatic patch, hemostasis to the tissue within approximately three minutes or less, the first plurality of subjects and the second plurality of subjects presenting a bleeding severity equal to or less than 3 at the bleeding site of the organ determined by a surface bleeding severity scale (SBSS).

Clause 38: The method of Clause 34, further comprising: reducing time to hemostatic control, by at least 63 seconds, by delivering the first hemostatic patch to the first plurality of subjects compared to the second plurality of subjects treated by delivering the second hemostatic patch.

Clause 39: The method of any of Clauses 34-38, further comprising: achieving approximately 100% hemostasis within approximately 45 seconds by delivering the first hemostatic patch to the first plurality of subjects.

Clause 40: The method of Clause 34, further comprising: restoring, by the first hemostatic patch, hemostasis to the tissue within approximately thirty seconds, the first plurality of subjects and the second plurality of subjects presenting a bleeding severity equal to or less than 2 at the bleeding site of the organ determined by a surface bleeding severity scale (SBSS).

Clause 41: The method of Clause 40, further comprising: reducing time to hemostatic control, by at least 150 seconds, by delivering the first hemostatic patch to the first plurality of subjects compared to the second plurality of subjects treated by delivering the second hemostatic patch.

Clause 42: The method of Clauses 40 or 41, further comprising: achieving approximately 100% hemostasis within approximately 30 seconds by delivering the first hemostatic patch to the first plurality of subjects.

Clause 43: The method of Clause 34, further comprising: restoring, by the first hemostatic patch, hemostasis to the tissue within approximately three minutes or less, the first plurality of subjects and the second plurality of subjects presenting a bleeding severity equal to or less than 4 at the bleeding site of the organ determined by a surface bleeding severity scale (SBSS).

Clause 44: The method of Clause 43, further comprising: reducing time to hemostatic control, by an average of 87 seconds, by delivering the first hemostatic patch to the first plurality of subjects compared to the second plurality of subjects treated by delivering the second hemostatic patch.

Clause 45: The method of Clause 43, further comprising: achieving approximately 100% hemostasis within approximately 93 seconds by delivering the first hemostatic patch to the first plurality of subjects.

Clause 46: The method of Clause 43, further comprising: before positioning the first hemostatic patch in contact with the tissue at a bleeding site, positioning at least a portion of the first hemostatic patch within a cavity of the bleeding site.

Clause 47: The method of Clause 46, further comprising: reducing time to hemostatic control, by at least 150 seconds, by delivering at least a portion of the first hemostatic patch within the cavity of the bleeding site of the first plurality of subjects compared to the second plurality of subjects treated by delivering the second hemostatic patch.

Clause 48: The method of Clauses 46 or 47, further comprising: achieving approximately 100% hemostasis within approximately 30 seconds by delivering at least a portion of the first hemostatic patch within the cavity of the bleeding site of the first plurality of subjects.

Clause 49: The method of Clause 34, further comprising: restoring, by the first hemostatic patch, hemostasis to the tissue within approximately three minutes or less, the first plurality of subjects and the second plurality of subjects presenting a bleeding severity equal to or less than 5 at the bleeding site of the organ determined by a surface bleeding severity scale (SBSS).

Clause 50: The method of Clause 49, further comprising: reducing time to hemostatic control, by an average of 188 seconds, by delivering the first hemostatic patch to the first plurality of subjects compared to the second plurality of subjects treated by delivering the second hemostatic patch.

Clause 51: The method of Clause 49, further comprising: achieving approximately 100% hemostasis within approximately 72 seconds by delivering the first hemostatic patch to the first plurality of subjects.

Clause 52: The method of Clause 49, further comprising: before positioning the first hemostatic patch in contact with the tissue at a bleeding site, positioning at least a portion of the first hemostatic patch within a cavity of the bleeding site.

Clause 53: The method of Clause 52, further comprising: reducing time to hemostatic control, by at least 150 seconds, by delivering at least a portion of the first hemostatic patch within the cavity of the bleeding site of the first plurality of subjects compared to the second plurality of subjects treated by delivering the second hemostatic patch.

Clause 54: The method of Clauses 52 or 53, further comprising: achieving approximately 100% hemostasis within approximately 30 seconds by delivering at least a portion of the first hemostatic patch within the cavity of the bleeding site of the first plurality of subjects.

Clause 55: The method of any of Clauses 34-54, further comprising: achieving approximately 100% hemostasis within 5 minutes or less by delivering the first hemostatic patch to the first plurality of subjects.

Clause 56: The method of any of Clauses 34-55, wherein the bleeding site is located in one of the following locations: liver, pancreas, spleen, stomach, gastrointestinal tract, kidney, bladder, reproductive organs, lungs, mediastinum, breast, lymph nodes, thymus, muscles, fat, heart, blood vessels, iliac arteries, carotid artery, vena cava, or brain.

Clause 57: A device for treating hemorrhage during a minimally invasive procedure, the device comprising a biocompatible, flexible, hemostatic patch comprising: a nucleophilic polymer carrying reactive nucleophilic groups, and an electrophilic polymer carrying at least three reactive electrophilic groups capable of reacting with the nucleophilic polymer and amine groups in tissue and blood; and wherein the hemostatic device is capable of being delivered through a trocar to an organ of a subject and restoring hemostasis to the organ within approximately three minutes or less by positioning the hemostatic device in contact with tissue at a bleeding site of the organ.

Clause 58: The device of Clause 57, wherein the electrophilic polymer is selected from polyoxazolines, polyethylene glycols, polyvinylpyrrolidones, polyurethanes and combinations thereof.

Clause 59: The device of Clauses 57 or 58, wherein the electrophilic polymer is a polyoxazoline.

Clause 60: The device of any of Clauses 57-59, wherein the reactive electrophilic groups are selected from the group consisting of carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, olefins, glycidyl ethers, carboxyl, succinimidyl esters, sulfo succinimidyl esters, maleimido (maleimidyl), ethenesulfonyl, imido esters, aceto acetate, halo acetal, orthopyridyl disulfide, dihydroxy-phenyl derivatives, vinyl, acrylate, acrylamide, iodoacetamide and combinations thereof.

Clause 61: The device of any of Clauses 57-60, wherein the hemostatic device is further configured to achieve hemostasis within approximately five minutes in 100% of subjects after positioning the hemostatic device in contact with tissue at the bleeding site of the organ in a respective subject.

Clause 62: The device of any of Clauses 57-60, wherein the hemostatic device is further configured to achieve hemostasis within approximately five minutes in 100% of subjects after positioning at least a portion of the hemostatic device within a cavity at the bleeding site of the organ in a respective subject.

Clause 63: The device of any of Clauses 57-60, wherein the hemostatic device is further configured to achieve hemostasis within approximately 30 seconds after positioning the hemostatic device in contact with tissue at the bleeding site of the organ in a respective subject, the subject presenting a bleeding severity equal to or less than 2 at the bleeding site of the organ determined by a surface bleeding severity scale (SBSS).

Clause 64: The device of any of Clauses 63, wherein the hemostatic device is further configured to achieve hemostasis within approximately 30 seconds after positioning at least a portion of the hemostatic device within a cavity at the bleeding site of the organ in a respective subject., the subject presenting a bleeding severity equal to or less than 2 at the bleeding site of the organ determined by a surface bleeding severity scale (SBSS).

Clause 65: The device of any of Clauses 57-60, wherein the hemostatic device is further configured to achieve hemostasis within approximately 45 seconds after positioning the hemostatic device in contact with tissue at the bleeding site of the organ in a respective subject, the subject presenting a bleeding severity equal to or less than 3 at the bleeding site of the organ determined by a surface bleeding severity scale (SBSS).

Clause 66: The device of any of Clauses 65, wherein the hemostatic device is further configured to achieve hemostasis within approximately 45 seconds after positioning at least a portion of the hemostatic device within a cavity at the bleeding site of the organ in a respective subject, the subject presenting a bleeding severity equal to or less than 3 at the bleeding site of the organ determined by a surface bleeding severity scale (SBSS).

Clause 67: The device of any of Clauses 57-60, wherein the hemostatic device is further configured to achieve hemostasis within approximately 72 seconds after positioning the hemostatic device in contact with tissue at the bleeding site of the organ in a respective subject, the subject presenting a bleeding severity equal to or less than 5 at the bleeding site of the organ determined by a surface bleeding severity scale (SBSS).

Clause 68: The device of any of Clauses 67, wherein the hemostatic device is further configured to achieve hemostasis within approximately 72 seconds after positioning at least a portion of the hemostatic device within a cavity at the bleeding site of the organ in a respective subject, the subject presenting a bleeding severity equal to or less than 5 at the bleeding site of the organ determined by a surface bleeding severity scale (SBSS).

Clause 69: The device of any of Clauses 57-68, wherein the bleeding site is located in one of the following locations: liver, pancreas, spleen, stomach, gastrointestinal tract, kidney, bladder, reproductive organs, lungs, mediastinum, breast, lymph nodes, thymus, muscles, fat, heart, blood vessels, iliac arteries, carotid artery, vena cava, or brain.

Clause 70: The device of any of Clauses 57-69, wherein the hemostatic device is further configured to adhere to the organ until at least a portion of the hemostatic device biodegrades within approximately 6 weeks.

Clause 71: The device of any of Clauses 57-70, wherein the hemostatic device is further configured to degrade after restoring hemostasis to the organ.

Clause 72: A biocompatible, flexible, hemostatic device for treating hemorrhage during a minimally invasive procedure, the hemostatic device comprising: a water-resistant cohesive fibrous carrier structure comprising: a three-dimensional interconnected interstitial space comprising a plurality of reactive polymer particles comprising an electrophilic polymer, and fibers comprising a nucleophilic polymer carrying reactive nucleophilic groups;

and wherein the hemostatic device is capable of being delivered through a trocar to an organ of a subject and restoring hemostasis to the organ within approximately three minutes or less by positioning the hemostatic device near or about a bleeding site of the organ.

Clause 73: The device of Clause 72, wherein the hemostatic device is further configured to achieve hemostasis within approximately five minutes in 100% of subjects after positioning the hemostatic device in contact with tissue at the bleeding site of the organ in a respective subject.

Clause 74: The device of Clause 72, wherein the hemostatic device is further configured to achieve hemostasis within approximately 30 seconds after positioning the hemostatic device in contact with tissue at the bleeding site of the organ in a respective subject, the subject presenting a bleeding severity equal to or less than 2 at the bleeding site of the organ determined by a surface bleeding severity scale (SBSS).

Clause 75: The device of Clause 72, wherein the hemostatic device is further configured to achieve hemostasis within approximately 45 seconds after positioning the hemostatic device in contact with tissue at the bleeding site of the organ in a respective subject, the subject presenting a bleeding severity equal to or less than 3 at the bleeding site of the organ determined by a surface bleeding severity scale (SBSS).

Clause 76: The device of Clause 72, wherein the hemostatic device is further configured to achieve hemostasis within approximately 93 seconds after positioning the hemostatic device in contact with tissue at the bleeding site of the organ in a respective subject, the subject presenting a bleeding severity equal to or less than 4 at the bleeding site of the organ determined by a surface bleeding severity scale (SBSS).

Clause 77: The device of Clause 72, wherein the hemostatic device is further configured to achieve hemostasis within approximately 72 seconds after positioning the hemostatic device in contact with tissue at the bleeding site of the organ in a respective subject, the subject presenting a bleeding severity equal to or less than 5 at the bleeding site of the organ determined by a surface bleeding severity scale (SBSS).

Clause 78: The device of Clause 72, wherein the bleeding site is located in one of the following locations: liver, pancreas, spleen, stomach, gastrointestinal tract, kidney, bladder, reproductive organs, lungs, mediastinum, breast, lymph nodes, thymus, muscles, fat, heart, blood vessels, iliac arteries, carotid artery, vena cava, or brain.

Clause 79: The device of Clause 72, wherein the hemostatic device is further configured to adhere to the organ until at least a portion of the hemostatic device biodegrades within approximately 6 weeks.

Clause 80: The device of Clause 79, wherein the hemostatic device is further configured to degrade after restoring hemostasis to the organ.

Clause 81: The device of Clause 72, the electrophilic polymer comprises at least three reactive electrophilic groups that are capable of reacting with the nucleophilic polymer and amine groups in tissue of the organ and blood.

Clause 82: The device of Clause 81, the reactive polymer particles comprising a diameter in a range of about 0.5 μm to about 100 μm and being present in an amount of at least 3% by weight of the fibrous carrier structure.

Clause 83: The device of Clause 72, wherein the fibrous carrier structure is a felt structure, a woven structure, or a knitted structure.

Clause 84: The device of Clause 78, wherein the electrophilic polymer is selected from polyoxazolines, polyethylene glycols, polyvinylpyrrolidones, polyurethanes and combinations thereof.

Clause 85: The device of Clause 84, wherein the electrophilic polymer is a polyoxazoline.

Clause 86: The device of Clause 81, wherein the reactive electrophilic groups are selected from the group consisting of carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, olefins, glycidyl ethers, carboxyl, succinimidyl esters, sulfo succinimidyl esters, maleimido (maleimidyl), ethenesulfonyl, imido esters, aceto acetate, halo acetal, orthopyridyl disulfide, dihydroxy-phenyl derivatives, vinyl, acrylate, acrylamide, iodoacetamide and combinations thereof.

Clause 87: The device of Clause 72, the hemostatic device comprising a molar ratio of electrophilic polymer to nucleophilic polymer ranging from about 1.0:0.10 to about 1.0:0.40.

Clause 88: The device of Clause 72, the hemostatic device capable of achieving hemostasis within about 10 seconds.

Clause 89: The device of any of Clauses 72-88, the hemostatic device further comprising a blue colorant.

The embodiments described above are cited by way of example, and the present invention is not limited by what has been particularly shown and described hereinabove. Rather, the scope of the invention includes both combinations and sub combinations of the various features described and illustrated hereinabove, as well as variations and modifications thereof which would occur to persons skilled in the art upon reading the foregoing description and which are not disclosed in the prior art.

What is claimed is:

1. A method for treating hemorrhage in a subject during a minimally invasive procedure, the method comprising:
delivering, through a trocar, a hemostatic patch near or about a bleeding site of an organ of a subject, the hemostatic patch comprising:
a fibrous carrier structure, and
reactive electrophilic groups capable of reacting with amine groups in tissue and blood;
positioning the hemostatic patch in contact with a tissue at the bleeding site; and
restoring hemostasis to the tissue within three minutes or less.

2. The method of claim 1, the hemostatic patch further comprising:
a three-dimensional interconnected interstitial space comprising a plurality of reactive polymer particles comprising:
an electrophilic polymer carrying the reactive electrophilic groups, and
a nucleophilic cross-linking agent that contains reactive nucleophilic groups that are capable of reacting with the reactive electrophilic groups of the electrophilic polymer under the formation of a covalent bond.

3. The method of claim 1, further comprising at least one of:
rolling the hemostatic patch into a delivery configuration;
unrolling the hemostatic patch into a treatment configuration;
applying pressure to the hemostatic patch positioned in contact with the tissue at the bleeding site, the pressure being applied for about 30 seconds; and
allowing degradation of the hemostatic patch after restoring hemostasis to the organ.

4. The method of claim 1, wherein at least one of:
the trocar comprises a diameter ranging from about 8 mm to about 16 mm;
the bleeding site is located in one of the following locations: liver, pancreas, spleen, stomach, gastrointestinal tract, kidney, bladder, reproductive organs, lungs, mediastinum, breast, lymph nodes, thymus, muscle, fat, heart, blood vessel, iliac artery, carotid artery, vena cava, or brain; and
the hemostatic patch is configured to fully degrade within approximately six weeks.

5. The method of claim 1, further comprising:
achieving, within approximately 30 seconds, hemostasis to the organ to a subject presenting a bleeding severity equal to or less than 2 at the bleeding site of the organ determined by a surface bleeding severity scale (SBSS).

6. The method of claim 1, further comprising:
achieving, within approximately 93 seconds, hemostasis to the organ to a subject presenting a bleeding severity equal to or less than 4 at the bleeding site of the organ determined by a surface bleeding severity scale (SBSS).

7. The method of claim 1, further comprising:
achieving, within approximately 72 seconds, hemostasis to the organ to a subject presenting a bleeding severity equal to or less than 5 at the bleeding site of the organ determined by a surface bleeding severity scale (SBSS).

8. The method of claim 1, further comprising providing the reactive electrophilic groups from the electrophilic polymer and the reactive nucleophilic groups from a nucleophilic polymer.

9. The method of claim 8, wherein at least one of:
the electrophilic polymer is selected from polyoxazolines, polyethylene glycols, polyvinylpyrrolidones, polyurethanes and combinations thereof;
the reactive electrophilic groups are selected from the group consisting of carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, olefins, glycidyl ethers, carboxyl, succinimidyl esters, sulfo succinimidyl esters, maleimido (maleimidyl), ethenesulfonyl, imido esters, aceto acetate, halo acetal, orthopyridyl disulfide, dihydroxy-phenyl derivatives, vinyl, acrylate, acrylamide, iodoacetamide and combinations thereof; and
the hemostatic patch comprising a molar ratio of electrophilic polymer to the nucleophilic polymer ranging from about 1.0:0.10 to about 1.0:0.40.

10. A method for treating hemorrhage in a subject during a minimally invasive procedure, the method comprising:
delivering, through a trocar, a hemostatic patch near or about a bleeding site of an organ of the subject, the hemostatic patch comprising:
a nucleophilic polymer carrying reactive nucleophilic groups, and
an electrophilic polymer carrying at least three reactive electrophilic groups capable of reacting with the nucleophilic polymer and amine groups in tissue and blood;
positioning the hemostatic patch in contact with a tissue at the bleeding site of the organ;
applying pressure to the hemostatic patch for approximately 30 seconds; and
restoring hemostasis of the organ within approximately three minutes or less.

11. The method of claim 10, further comprising at least one of:
reducing, by at least 40%, time of arterial clamping in a first plurality of subjects after positioning the hemostatic patch in contact with the tissue at the bleeding site of the organ and applying pressure for approximately 30 seconds in a respective subject, compared to a second plurality of subjects treated with sutures,
reducing, by at least 300%, time to hemostatic control in a first plurality of subjects after positioning the hemostatic patch in contact with the tissue at the bleeding site of the organ and applying pressure for approximately 30 seconds in a respective subject, compared to a second plurality of subjects treated with sutures;
reducing, by at least 1,142%, time of active bleeding in a first plurality of subjects after positioning the hemostatic patch in contact with the tissue at the bleeding site of the organ and applying pressure for approximately 30 seconds in a respective subject, compared to a second plurality of subjects treated with sutures; and
reducing, by at least 227%, total procedure time for a first plurality of subjects after positioning the hemostatic patch in contact with the tissue at the bleeding site of the organ and applying pressure for approximately 30 seconds in a respective subject, compared to a second plurality of subjects treated with sutures,
wherein the first plurality of subjects presents an average bleeding severity equal to or less than 3 at the bleeding site of the organ determined by a surface bleeding severity scale (SBSS).

12. The method of claim 10, further comprising:
providing a sheet of water-resistant cohesive fibrous carrier structure comprising a three-dimensional interconnected interstitial space and fibers comprising the nucleophilic polymer carrying reactive nucleophilic groups;
providing reactive polymer particles comprising the electrophilic polymer comprising at least three reactive electrophilic groups that are capable of reacting with both amine groups in tissue and blood and with the reactive nucleophilic groups of the nucleophilic polymer to form covalent bonds; and
distributing the reactive polymer particles within the interstitial space of the fibrous carrier structure in an amount of at least 3% by weight of the fibrous carrier structure for form the hemostatic patch.

13. The method of claim 10, further comprising at least one of:
reducing, by delivering the hemostatic patch to a first plurality of subjects, time to hemostatic control of active bleeding from the bleeding site of the organ compared to a second plurality of subjects treated by suturing the bleeding site of the organ;
reducing, by delivering the hemostatic patch to a first plurality of subjects, time of active bleeding from the bleeding site of the organ during hemostatic control compared to a second plurality of subjects treated by suturing the bleeding site of the organ; and
reducing, by delivering the hemostatic patch to a first plurality of subjects, total procedure time of treating hemorrhage in the first plurality of subjects compared to a second plurality of subjects treated by suturing the bleeding site of the organ.

14. The method of claim 13, further comprising at least one of:
reducing time to hemostatic control by at least 1 minute and 7 seconds, by delivering the hemostatic patch to the first plurality of subjects;
reducing time of active bleeding from the bleeding site of the organ during hemostatic control by at least 4 seconds, by delivering the hemostatic patch to the first plurality of subjects; and
reducing total procedure time by at least 1 minute and 11 seconds, by delivering the hemostatic patch to the first plurality of subjects.

15. The method of claim 14, further comprising at least one of:
reducing time to hemostatic control by an average of approximately 6 minutes and 28 seconds, by delivering the hemostatic patch to the first plurality of subjects;
reducing time of active bleeding from the bleeding site of the organ during hemostatic control by an average of approximately 5 minutes and 23 seconds, by delivering the hemostatic patch to the first plurality of subjects; and
reducing total procedure time by an average of approximately 6 minutes and 59 seconds, by delivering the hemostatic patch to the first plurality of subjects.

16. A method for treating hemorrhage in a subject during a minimally invasive procedure, the method comprising:
delivering, through a trocar, a first hemostatic patch near or about a bleeding site of an organ of a respective subject in a first plurality of subjects, the first hemostatic patch comprising:
a nucleophilic polymer carrying reactive nucleophilic groups, and
an electrophilic polymer carrying at least three reactive electrophilic groups capable of reacting with the nucleophilic polymer and amine groups in tissue and blood;
positioning the first hemostatic patch in contact with a tissue at the bleeding site;
applying pressure to the first hemostatic patch for approximately 30 seconds; and
reducing time to hemostatic control of active bleeding from the bleeding site of the organ by delivering the first hemostatic patch to the first plurality of subjects compared to a second plurality of subjects treated by delivering a second hemostatic patch.

17. The method of claim 16, further comprising at least one of:
restoring, by the first hemostatic patch, hemostasis to the tissue within approximately three minutes or less, the first plurality of subjects and the second plurality of subjects presenting a bleeding severity equal to or less than 3 at the bleeding site of the organ determined by a surface bleeding severity scale (SBSS);
reducing time to hemostatic control, by at least 63 seconds, by delivering the first hemostatic patch to the first plurality of subjects compared to the second plurality of subjects treated by delivering the second hemostatic patch; and
achieving approximately 100% hemostasis within approximately 45 seconds by delivering the first hemostatic patch to the first plurality of subjects.

18. The method of claim 16, further comprising at least one of:
restoring, by the first hemostatic patch, hemostasis to the tissue within approximately thirty seconds, the first plurality of subjects and the second plurality of subjects presenting a bleeding severity equal to or less than 2 at the bleeding site of the organ determined by a surface bleeding severity scale (SBSS);
reducing time to hemostatic control, by at least 150 seconds, by delivering the first hemostatic patch to the first plurality of subjects compared to the second plurality of subjects treated by delivering the second hemostatic patch; and
achieving approximately 100% hemostasis within approximately 30 seconds by delivering the first hemostatic patch to the first plurality of subjects.

19. The method of claim 16, further comprising at least one of:
restoring, by the first hemostatic patch, hemostasis to the tissue within approximately three minutes or less, the first plurality of subjects and the second plurality of subjects presenting a bleeding severity equal to or less than 4 at the bleeding site of the organ determined by a surface bleeding severity scale (SBSS);
reducing time to hemostatic control, by an average of 87 seconds, by delivering the first hemostatic patch to the first plurality of subjects compared to the second plurality of subjects treated by delivering the second hemostatic patch; and
achieving approximately 100% hemostasis within approximately 93 seconds by delivering the first hemostatic patch to the first plurality of subjects.

20. The method of claim 16, further comprising at least one of:
before positioning the first hemostatic patch in contact with the tissue at a bleeding site, positioning at least a portion of the first hemostatic patch within a cavity of the bleeding site;
reducing time to hemostatic control, by at least 150 seconds, by delivering at least a portion of the first hemostatic patch within the cavity of the bleeding site of the first plurality of subjects compared to the second plurality of subjects treated by delivering the second hemostatic patch; and
achieving approximately 100% hemostasis within approximately 30 seconds by delivering at least a portion of the first hemostatic patch within the cavity of the bleeding site of the first plurality of subjects.
